(19) United States
(12) Patent Application Publication
(10) Pub. No.: US 2025/0236646 A1
(43) Pub. Date: Jul. 24, 2025

(54) TRUNCATED VARICELLA-ZOSTER VIRUS ENVELOPE GLYCOPROTEIN GE

(71) Applicants: XIAMEN UNIVERSITY, Xiamen, Fujian (CN); XIAMEN INNOVAX BIOTECH CO., LTD., Xiamen, Fujian (CN)

(72) Inventors: Shaowei LI, Xiamen, Fujian (CN); Tingting CHEN, Xiamen, Fujian (CN); Liqin LIU, Xiamen, Fujian (CN); Lizhi ZHOU, Xiamen, Fujian (CN); Tingting LI, Xiamen, Fujian (CN); Ying GU, Xiamen, Fujian (CN); Tong CHENG, Xiamen, Fujian (CN); Ningshao XIA, Xiamen, Fujian (CN)

(21) Appl. No.: 18/849,324

(22) PCT Filed: Mar. 21, 2023
(86) PCT No.: PCT/CN2023/082698

§ 371 Date: Sep. 20, 2024

(30) Foreign Application Priority Data
Mar. 21, 2022 (CN) 202210277819.7

(51) Int. Cl. C07K 14/005; A61K 39/00; A61P 37/04

(52) U.S. Cl. CPC C07K 14/005; A61P 37/04; A61K 39/00; C12N 2710/16722; C12N 2710/16734; C12N 2710/16771

(57) ABSTRACT

The present invention relates to the field of immunology and the field of molecular virology, in particular to the field of prevention and treatment of varicella-zoster viruses. Specifically, the present invention relates to a truncated varicella-zoster virus gE protein (or a variant thereof) capable of be soluble expression in an Escherichia coli expression system, and use thereof in preventing and/or treating varicella-zoster virus infections.

### TECHNICAL FIELD

The present application relates to the fields of immunology and molecular virology, in particular to the prevention and treatment of varicella-zoster virus. Specifically, the present application relates to a truncated varicella-zoster virus gE protein (or its variant) that can be expressed in a soluble form in an Escherichia coli expression system, and its use in the prevention and/or treatment varicella-zoster virus infection.

### BACKGROUND ART

Varicella-zoster virus (VZV), also known as human herpesvirus 3 (HHV-3), belongs to the a subfamily of the herpesviridae family, and is a type of envelope-containing double-stranded DNA virus. The VZV viral genome is about 12.5 kb and contains about 70 open reading frames. VZV particle is spherical in shape, about 150 to 200 nm in diameter, and comprises four parts: virus DNA, capsid, tegument protein and envelope protein radially outward from sphere center. The outer surface of the virus is an envelope formed by a variety of glycoproteins and lipids, in which the main glycoproteins are gB, gC, gE, gH, gL, gI, etc., and they play an important role in identifying host cells, stimulating immune responses, and enhancing the stability of virus particles.

VZV infection can be divided into primary infection and recurrent infection. Primary infection manifests as varicella (chickenpox), during which VZV mainly enters the body through respiratory droplets or skin contact of infected individuals, causing primary mucosal epithelial infection, spreading in the body through the blood or lymphatic system, causing viremia, spreading to the skin of the whole body, and manifesting as maculopapular rash and vesicular rash. Varicella is more common in children and is generally self-limited (except for immunodeficient children), and its symptoms are relatively mild, but varicella in adult is often accompanied by viral pneumonia and has a high mortality rate. After the host recovers, a small amount of VZV will still remain in the sensory ganglion cells of the dorsal root ganglia of spinal cord or the cranial nerves. When the host undergoes hypoimmunity or stimulated to a certain extent, the VZV lurking in the host will be activated and induce herpes zoster, causing severe pain and complications such as postherpetic neuralgia (PHN). The incidence of herpes zoster increases with age, seriously affecting the daily life of the elderly.

Antiviral drugs and vaccines are the best choice for treating or preventing VZV infection. Vaccination with related vaccines is also the best treatment method that can effectively relieve symptoms such as papules and postherpetic neuralgia. The live-attenuated vaccine based on the Oka strain can be used to prevent varicella and herpes zoster, but it has disadvantages such as inapplicability in immunocompromised patients, limited effect in preventing herpes zoster, and the risk of viral latency. Compared to live-attenuated vaccines, subunit vaccines have advantages such as high safety and low production cost. Subunit vaccines with VZV envelope glycoprotein gE as antigen have achieved significant positive outcomes in the treatment of elderly patients, the humoral and cellular immune responses stimulated thereby in the organism are stronger than those induced by live-attenuated vaccines, making them a new avenue for the development of varicella and herpes zoster vaccines.

In vaccine research and development, safety, protection effect and economic factors should be taken into account. Commonly used antigen protein expression systems can be divided into eukaryotic expression systems and prokaryotic expression systems. The gE protein, when expressed in a eukaryotic expression system, sustains minimal natural conformational damage, ensuring the correct conformation and epitope presentation. However, the baculovirus expression system, yeast expression system and CHO expression system used in the current eukaryotic expression systems all have defects such as high culture cost, which poses significant challenges for large-scale industrial production. Shingrix, a recombinant herpes zoster vaccine based on AS01_{B} adjuvant, which is currently on the market, uses the CHO expression system to produce gE extracellular segment protein as a vaccine antigen. The price of this product is relatively high, which affects its wide application.

Among the prokaryotic expression systems, the Escherichia coli expression system has the advantages of fast growth rate and low culture cost, and is an effective tool for producing recombinant proteins. However, the viral proteins expressed in the Escherichia coli expression system often lose their correct natural conformation and are expressed in the form of inclusion bodies within the precipitate. At present, the renaturation of proteins expressed in inclusion bodies is still a global problem. Folding errors often occur during the renaturation process, resulting in protein precipitation, or local conformation errors after folding lead to reduced protein activity and decreased immunogenicity. Although the complete extracellular segment of gE protein can also be expressed in the Escherichia coli lysis supernatant in a soluble form with correct conformation, it is also quite difficult to purify the gE protein from the various soluble proteins in the Escherichia coli lysis supernatant. It often necessitates the use of fusion strategy and affinity chromatography. The above methods often require expensive enzymes and are difficult to carry out industrial production. At present, there is no literature reporting the high-efficiency soluble expression of the gE protein extracellular segment in Escherichia coli and the purification of the solubly expressed protein as a VZV vaccine antigen.

Therefore, there is still a need in the art for a low-cost gE protein and a vaccine thereof that can induce a protective antibody against VZV, so as to make the large-scale industrial production of varicella and herpes zoster vaccines possible.

### CONTENTS OF THE APPLICATION

The inventors of the present application screened out a series of truncated VZV gE proteins through a large number of experiments. The truncated proteins, while retaining most of the immune epitopes, can be expressed in Escherichia coli with a greater degree of solubility than the complete extracellular region of gE protein, and is easier to purify, while also well retaining the antigenicity of the VZV gE protein.

### Truncated gE Protein or Variant Thereof

Therefore, in one aspect, the present application provides a truncated varicella-zoster virus (VZV) gE protein or variant thereof, wherein the truncated VZV gE protein has a truncation at the C-terminal of 75-445 amino acids as compared to the wild-type VZV gE protein; the variant has an amino acid sequence identity of at least 90%, such as at least 95%, at least 96%, at least 97%, at least 98%, at least 99%, or has a substitution (preferably, a conservative substitution), addition or deletion of one or several (e.g., 1, 2, 3, 4, 5, 6, 7, 8 or 9) amino acids, as compared to the truncated VZV gE protein, and retains the biological function of the truncated VZV gE protein (e.g., the ability to induce a neutralizing antibody against VZV, and/or soluble expression in Escherichia coli).

In certain embodiments, as compared to the wild-type VZV gE protein, the truncated VZV gE protein has a truncation at the C-terminal of at most 445 amino acids, such as at most 442, at most 440, at most 430, at most 420, at most 410, at most 400, at most 390, at most 380, at most 370, at most 360, at most 350, at most 340, at most 330, at most 325 or at most 320 amino acids, for example, at most 330 amino acids; and/or, has a truncation at the C-terminal of at least 75 amino acids, such as at least 77, at least 80, at least 85, at least 90, at least 100, at least 110, at least 120, at least 130, at least 140, at least 150, at least 160, at least 170, at least 180, at least 190, at least 193, at least 200, at least 210, at least 220, at least 222, at least 230, at least 240, at least 248, at least 250, at least 260 or at least 265 amino acids, for example at least 260 amino acids.

In some embodiments, as compared to the wild-type VZV gE protein, the truncated VZV gE protein has a truncation at the C-terminal of 80-445, 190-445, 220-445, 245-445, 260-445, 75-330, 80-330, 190-330, 220-330, 245-330, 260-330, 75-310, 80-310, 190-310, 220-310, 245-310, 260-310, 75-300, 80-300, 190-300, 220-300, 245-300, 260-300, 75-270, 80-270, 190-270, 220-270, 245-270, 77-442, 85-442, 193-442, 222-442, 248-442, 265-442, 77-320, 85-320, 193-320, 222-320, 248-320, 265-320, 77-303, 85-303, 193-303, 222-303, 248-303, 265-303, 77-293, 85-293, 193-293, 222-293, 248-293, 265-293, 77-265, 85-265, 193-265, 222-265 or 248-265 amino acids.

In certain embodiments, as compared to the wild-type VZV gE protein, the truncated VZV gE protein has no truncation at the N-terminal.

In certain embodiments, as compared to the wild-type VZV gE protein, the truncated VZV gE protein has a truncation at the N-terminal of 1-170 amino acids.

In certain embodiments, as compared to the wild-type VZV gE protein, the truncated VZV gE protein has a truncation at the N-terminal of at most 170 amino acids, such as at most 165, at most 160, at most 155, at most 150, at most 145, at most 140, at most 139, at most 135, at most 130, or at most 127 amino acids, for example at most 130 amino acids; and/or, has a truncation at the N-terminal of at least 1 amino acid, such as at least 5, at least 10, at least 15, at least 20, at least 25, or at least 30 amino acids, for example at least 30 amino acids.

In certain embodiments, as compared to the wild-type VZV gE protein, the truncated VZV gE protein has a truncation at the N-terminal of 20-170, 1-155, 20-155, 1-140, 20-140, 1-130, 20-130, 1-85, 20-85, 1-75, 20-75, 1-30, 20-30, 30-165, 30-152, 30-139, 30-127, 30-80, 30-73, 30-75, 30-85, 30-130, 30-140, 30-155 or 30-170 amino acids.

In certain embodiments, as compared to the wild-type VZV gE protein, the truncated VZV gE protein has a truncation at the C-terminal of 260-330 amino acids, such as, 260-310, 260-300, 265-320, 265-303, 265-293 amino acids.

In certain embodiments, as compared to the wild-type VZV gE protein, the truncated VZV gE protein has no truncation at the N-terminal or has a truncation at the N-terminal of 1-130 amino acids, for example, 20-130, 20-85, 20-75, 20-30, 30-127, 30-80, 30-73, 30-75, 30-85, 30-130 amino acids. In certain embodiments, the truncated VZV gE protein has no truncation at the N-terminal or has a truncation at the N-terminal of 30-130 amino acids.

In certain embodiments, as compared to the wild-type VZV gE protein, the truncated VZV gE protein has a truncation at the C-terminal of 260-330 amino acids, for example, 260-310, 260-300, 265-320, 265-303, 265-293 amino acids; has no truncation at the N-terminal or has a truncation at the N-terminal of 20-130 amino acids, for example, 20-85, 20-75, 20-30, 30-127, 30-80, 30-73, 30-75, 30-85, 30-130 amino acids. In certain embodiments, the truncated VZV gE protein has no truncation at the N-terminal or has a truncation at the N-terminal of 30-130 amino acids.

In certain embodiments, as compared to the wild-type VZV gE protein, the truncated VZV gE protein has a truncation at the C-terminal of 77, 85, 193, 222, 248, 265, 293, 303 or 320 amino acids; has no truncation at the N-terminal or has a truncation at the N-terminal of 30, 73, 80, 127 or 139 amino acids.

In certain embodiments, as compared to the wild-type VZV gE protein, the truncated VZV gE protein has a truncation at the C-terminal of 265, 293, 303 or 320 amino acids; has a truncation at the N-terminal of 30, 73, 80 or 127 amino acids.

In certain embodiments, as compared to the wild-type VZV gE protein, the truncated VZV gE protein has a truncation at the C-terminal of 265 amino acids; has a truncation at the N-terminal of 30 amino acids.

In certain embodiments, the wild-type VZV gE protein has an amino acid sequence as shown in SEQ ID NO: 19, or a sequence having a sequence identity of at least 90%, at least 95%, at least 96%, at least 97%, at least 98%, at least 99% as compared thereto.

In certain embodiments, the truncated VZV gE protein has an amino acid sequence as shown in any one of SEQ ID NOs: 1 to 6, 20 to 30.

In certain embodiments, the truncated VZV gE protein is capable of binding to an anion exchange chromatography medium (e.g., Q Sepharose 4 Fast Flow) in a solution with a pH of 7.5 to 8.5 (e.g., pH 8.0).

In certain embodiments, the truncated VZV gE protein is capable of binding to an anion exchange chromatography medium (e.g., Q Sepharose 4 Fast Flow) in a solution with a pH of 7.5 to 8.5 (e.g., pH 8.0) and a salt concentration of 0-300 mM (e.g., 0-200 mM, 0-50 mM).

### Isolated Nucleic Acid Molecule

In another aspect, the present application provides an isolated nucleic acid molecule, which encodes the truncated VZV gE protein or variant thereof as described above.

In certain embodiments, the nucleotide sequence encoding the truncated VZV gE protein or variant thereof comprised in the isolated nucleic acid molecule is codon-optimized according to the codon preference of the host cell (e.g., Escherichia coli) or unoptimized.

### Vector

In another aspect, the present application provides a vector, which comprises the isolated nucleic acid molecule as described above.

### Host Cell

In another aspect, the present application provides a host cell, which comprises the isolated nucleic acid molecule or the vector as described above.

In some embodiments, the host cell is selected from prokaryotic cells (e.g., Escherichia coli cells) and eukaryotic cells (e.g., yeast cells, insect cells, plant cells, mammalian cells). In some embodiments, the host cell is a microorganism.

In some embodiments, the host cell is an Escherichia coli cell.

### Preparation Method

In another aspect, the present application also provides a method for preparing the truncated VZV gE protein or variant thereof as described above, comprising culturing the host cell as described above under a condition that allows protein expression, and recovering the truncated VZV gE protein or variant thereof from a culture of the cultured host cell.

In certain embodiments, the method comprises recovering the truncated VZV gE protein or variant thereof from a culture supernatant or lysis supernatant of the cultured host cell.

In certain embodiments, the host cell is Escherichia coli.

In certain embodiments of the method, the recovery step comprises protein purification.

In certain embodiments, the protein purification comprises performing an ion exchange chromatography, hydroxyapatite chromatography, and/or hydrophobic interaction chromatography.

In certain embodiments, the ion exchange chromatography comprises:
a) allowing the truncated VZV gE protein or variant thereof to bind to an anion exchange chromatography medium (e.g., Q Sepharose 4 Fast Flow) in a solution with a pH of 7.5 to 8.5 (e.g., pH 8.0) and a salt concentration of 0 mM to 300 mM (e.g., 0 mM to 200 mM, 0 mM to 50 mM);
b) performing a gradient elution by gradually increasing the salt concentration of the solution;
c) collecting an elution fraction containing the truncated VZV gE protein or variant thereof when the solution salt concentration is within the range of 350 mM to 450 mM (e.g., at 400 mM).

### Immunogenic Composition

In another aspect, the present application also provides an immunogenic composition, which comprises the truncated VZV gE protein or variant thereof as described above, and optionally a pharmaceutically acceptable carrier and/or excipient (e.g., adjuvant).

In certain embodiments, the immunogenic composition comprises the truncated VZV gE protein or variant thereof as described above and an adjuvant, wherein the adjuvant is selected from the group consisting of: risedronate adjuvant (e.g., zinc-aluminum hybrid adjuvant containing risedronate sodium), aluminum adjuvant (e.g., aluminum hydroxide adjuvant, aluminum phosphate adjuvant), zinc-aluminum hybrid adjuvant (e.g., FH002C), Freund's adjuvant, oil emulsion adjuvant, cytokine, TLR agonist, CpG adjuvant, liposome, AS01_{B} adjuvant, zoledronate sodium, monophosphoryl lipid A (MPL), cholesterol-containing liposome and combination thereof. In certain embodiments, the adjuvant is selected from the group consisting of: risedronate adjuvant (e.g., zinc-aluminum hybrid adjuvant containing risedronate sodium), aluminum adjuvant (e.g., aluminum hydroxide adjuvant, aluminum phosphate adjuvant), AS01_{B} adjuvant and combination thereof.

In certain embodiments, the immunogenic composition comprises the truncated VZV gE protein or variant thereof as described above and AS01_{B} adjuvant.

In certain embodiments, the immunogenic composition is a vaccine.

The immunogenic composition as described above can be formulated into any dosage form known in the medical field, for example, tablets, pills, suspensions, emulsions, solutions, gels, capsules, powders, granules, elixirs, lozenges, suppositories, injections (including injections, sterile powders for injection and concentrated solutions for injection), inhalants, sprays, etc. The preferred dosage form depends on the intended mode of administration and therapeutic use. The immunogenic composition of the present application should be sterile and stable under production and storage conditions. A preferred dosage form is an injection. Such an injection can be a sterile injection solution. In addition, the sterile injection solution can be prepared as a sterile lyophilized powder (e.g., by vacuum drying or freeze drying) for easy storage and use. Such a sterile lyophilized powder can be dispersed in a suitable carrier before use, such as water for injection (WFI), bacteriostatic water for injection (BWFI), sodium chloride solution (e.g., 0.9% (w/v) NaCl), glucose solution (e.g., 5% glucose), solution containing surfactant (e.g., a solution containing 0.01% polysorbate 20), pH buffer solution (e.g., phosphate buffer solution), Ringer's solution, and any combination thereof.

The immunogenic composition described above can be administered by any suitable method known in the art, including, but not limited to, oral, buccal, sublingual, ocular, local, parenteral, rectal, intrathecal, intracytoplasmic reticulum, inguinal, intravesical, topical (e.g., powder, ointment or drops), or nasal route. However, for many therapeutic uses, the preferred route/mode of administration is parenteral administration (e.g., intravenous or bolus injection, subcutaneous injection, intraperitoneal injection, intramuscular injection). The skilled person will understand that the route and/or mode of administration will vary depending on the intended purpose.

The immunogenic composition as described above should be administered in an amount sufficient to induce an immune response against VZV. The appropriate amount of the immunogen can be determined based on the specific disease or condition to be treated or prevented, the severity, the age of the subject, and other personal attributes of the specific subject (e.g., the general state of health of the subject and the robustness of the subject's immune system). The determination of the effective amount is also guided by animal model studies, followed by human clinical trials, and by an administration regimen that significantly reduces the occurrence or severity of the target disease symptoms or conditions in the subject.

### Use in Manufacture of an Immunogenic Composition

In another aspect, the present application also provides a use of the truncated VZV gE protein or variant thereof, or the isolated nucleic acid molecule, or the vector, or the host cell as described above in the manufacture of an immunogenic composition, and the immunogenic composition is used for inducing an immune response against VZV in a subject and/or for preventing and/or treating a VZV infection or a disease associated with VZV infection in a subject.

In certain embodiments, the immunogenic composition is a vaccine.

In certain embodiments, the VZV infection is a primary infection or a recurrent infection with VZV.

In certain embodiments, the disease associated with VZV infection is selected from the group consisting of: herpes zoster, varicella, and complication thereof (e.g., neuropathic pain, pneumonia, encephalomyelitis, conjunctivitis).

In certain embodiments, the disease associated with VZV infection is selected from the group consisting of: herpes zoster, varicella, and postherpetic neuralgia.

In certain embodiments, the subject is a mammal, such as a human.

Method for Preventing and/or Treating Disease

In another aspect, the present application also provides a method for inducing an immune response against VZV in a subject and/or for preventing and/or treating a VZV infection or a disease associated with VZV infection in a subject, comprising: administering an effective amount of the truncated VZV gE protein or variant thereof, the isolated nucleic acid molecule, the vector, the host cell or the immunogenic composition as described above to the subject in need thereof.

In certain embodiments, the VZV infection is a primary infection or a recurrent infection of VZV.

In certain embodiments, the disease associated with VZV infection is selected from the group consisting of: herpes zoster, varicella, and complications thereof (e.g., neuropathic pain, pneumonia, encephalomyelitis, conjunctivitis).

In certain embodiments, the disease associated with VZV infection is selected from the group consisting of: herpes zoster, varicella, and postherpetic neuralgia.

In certain embodiments, the subject is a mammal, such as a human.

### Detection Method

In another aspect, the present application also provides a method for detecting the presence of a VZV gE protein-specific antibody in a sample, which comprises using the truncated VZV gE protein or variant thereof as described above.

In certain embodiments, the method is an immunological assay, such as immunoblotting, enzyme immunoassay (e.g., ELISA), chemiluminescent immunoassay, fluorescent immunoassay, or radioimmunoassay.

In certain embodiments, the method comprises: (1) contacting the sample with the truncated VZV gE protein or variant thereof as described above; (2) detecting the formation of a protein-antibody immune complex or detecting an amount of the immune complex; the formation of the immune complex indicates the presence of VZV gE protein-specific antibody in the sample.

In some embodiments, the method further comprises using a second antibody with a detectable label (e.g., an enzyme (e.g., horseradish peroxidase or alkaline phosphatase), a chemiluminescent agent (e.g., acridinium ester compound, luminol and derivative thereof, or ruthenium derivative), a fluorescent dye (e.g., fluorescein or fluorescent protein), a radionuclide, or biotin) to detect the presence of the VZV gE protein-specific antibody in the sample.

In some embodiments, the second antibody is specific for a constant region contained in an antibody of the species (e.g., human) from which the sample to be tested is derived.

In some embodiments, the second antibody is an anti-immunoglobulin (e.g., human immunoglobulin) antibody, such as an anti-IgG antibody.

In some embodiments, the sample is a body fluid sample (e.g., whole blood, plasma, serum, salivary excretion, or urine) from a subject (e.g., a mammal, preferably a human).

### Use in Manufacture of Detection Reagent

In another aspect, the present application also provides a use of the truncated VZV gE protein or variant thereof, or the isolated nucleic acid molecule, or the vector, or the host cell as described above in the manufacture a detection reagent, wherein the detection reagent is used to detect the presence of a VZV gE protein-specific antibody in a sample.

In certain embodiments, the detection reagent detects the presence of VZV gE protein-specific antibody in the sample by the detection method as described above.

In certain embodiments, the sample is a body fluid sample (e.g., whole blood, plasma, serum, salivary excretion, or urine) from a subject (e.g., a mammal, preferably a human).

### Kit

In another aspect, the present application also provides a kit, which comprises the truncated VZV gE protein or variant thereof, or the isolated nucleic acid molecule, or the vector, or the host cell as described above.

In certain embodiments, the kit comprises the truncated VZV gE protein or variant thereof as described above, and a second antibody, wherein the second antibody is as defined above.

### DEFINITION OF TERMS

In the present application, unless otherwise specified, the scientific and technical terms used herein have the meanings commonly understood by those skilled in the art. In addition, the virology, biochemistry, and immunology laboratory operation steps used herein are all routine steps widely used in the corresponding fields. At the same time, in order to better understand the present application, the definitions and explanations of relevant terms are provided below.

When the terms “for example”, “e.g.”, “such as”, “including”, “comprising” or variations thereof are used herein, these terms will not be considered as restrictive terms, but will be interpreted as meaning “but not limited to” or “not limited to”.

Unless otherwise specified herein or clearly contradicted by the context, the terms “a”, “an” and “the” and similar referents should be interpreted as covering the singular and plural in the context of describing the present application (especially in the context of the following claims).

As used herein, the term “VZV” is an abbreviation for “varicella-zoster virus”, which is a DNA virus.

As used herein, the terms “gE protein”, “gE”, and “glycoprotein gE” refer to a kind of envelope glycoprotein of VZV, which have the same meaning and can be used interchangeably. The specific amino acid sequence of the wild-type gE protein can be obtained from a public database (e.g., GenBank database), for example, the amino acid sequence shown in GenBank accession number CAA27951.1 (Dumas strain), AAT07825.1 (VZV-MSP strain) or AAT07749.1″ (BC strain).

In the present application, the expression “truncation at the N-terminal of X amino acids” for a protein means that: (1) the amino acid residues at positions 1 to X at the N-terminal of the protein are deleted, or (2) the amino acid residues at positions 1 to X at the N-terminal of the protein are replaced by the amino acid residue (e.g., methionine residue) encoded by the start codon (for initiating protein translation). It is known to those skilled in the art that during the translation of mRNA, due to the effect of the start codon, the amino acid at position 1 of the generated polypeptide chain is usually the amino acid encoded by the start codon (e.g., methionine (M)). Therefore, the residue at position 1 of the truncated protein having truncation of X amino acids at the N-terminal of the present application can be the amino acid at position “X+1” of the full-length protein, or the residue at position 1 can be the amino acid encoded by the start codon that is additionally included before the amino acid at position “X+1”. For example, the VZV gE protein having a truncation of 30 amino acids at the N-terminal includes an amino acid sequence selected from the following: (1) an amino acid sequence having a deletion of the amino acid residues at positions 1 to 30 as compared to the wild-type VZV gE protein, or (2) an amino acid sequence obtained by replacing the amino acid residues at positions 1 to 30 at the N-terminal of the wild-type VZV gE protein with the amino acid encoded by the start codon (e.g., methionine).

In the present application, the expression “truncation at the C-terminal of X amino acids” for a protein refers to a deletion of X amino acid residues closest to the C-terminal of the protein. For example, a VZV gE protein having a truncation of 265 amino acids at the C-terminal refers to a VZV gE protein having a deletion of 265 amino acid residues closest to the C-terminal.

In the present application, when referring to the amino acid sequence of the wild-type gE protein, the description is made with reference to the sequence as shown in SEQ ID NO: 19. For example, the expression “amino acid residues at positions 1 to 30 of the wild-type gE protein” refers to the amino acid residues at positions 1 to 30 of the polypeptide as shown in SEQ ID NO: 19. However, those skilled in the art understand that VZV may include multiple isolates, and there may be differences between the amino acid sequences of the gE proteins of various isolates. Further, those skilled in the art understand that, although there may be sequence differences, the gE proteins of different isolates of VZV have extremely high identity in amino acid sequence (usually higher than 95%, such as higher than 96%, higher than 97%, higher than 98%, or higher than 99%), and have substantially the same biological function. Therefore, in the present application, the term “wild-type gE protein” includes not only the protein as shown in SEQ ID NO: 19, but also the gE proteins of various VZV isolates. And, when describing the sequence fragment of the wild-type gE protein, it includes not only the sequence fragment of SEQ ID NO: 19, but also the corresponding sequence fragments in the gE proteins of various VZV isolates. For example, the expression “amino acid residues at positions 1 to 30 of the wild-type gE protein” includes the amino acid residues at positions 1 to 30 of SEQ ID NO: 19, and the corresponding fragments in the gE proteins of various VZV isolates.

According to the present application, the expression “corresponding sequence fragment” or “corresponding amino acid position” refers to the fragment or amino acid site/residue at the equivalent position in the compared sequences when the sequences are optimally aligned, i.e. when the sequences are aligned to obtain the highest percentage identity.

According to the present application, the term “variant” refers to a protein which amino acid sequence has an identity of at least 90%, such as at least 95%, at least 96%, at least 97%, at least 98%, at least 99%, or has a substitution (preferably conservative substitution), addition or deletion of one or several (e.g., 1, 2, 3, 4, 5, 6, 7, 8 or 9) amino acids, as compared to the amino acid sequence of the truncated VZV gE protein of the present application, and retains the essential properties of the truncated VZV gE protein. Here, the term “essential properties” may be one or more of the following properties: ability to specifically bind to an anti-gE monoclonal antibody; ability to induce a neutralizing antibody against VZV; ability to undergo soluble expression in Escherichia coli.

As used herein, the term “identity” is used to refer to the matching of sequences between two polypeptides or between two nucleic acids. When a position in both compared sequences is occupied by the same nucleotide or amino acid residues (e.g., a position in each of the two DNA molecules is occupied by adenine nucleotide, or a position in each of the two polypeptides is occupied by lysine), then the molecules are identical at that position. The “percent identity” between two sequences is a function of the number of matching positions shared by the two sequences divided by the number of positions compared x 100. For example, if 6 out of 10 positions in two sequences match, then the two sequences have an identity of 60%. For example, the DNA sequences CTGACT and CAGGTT share 50% identity (3 out of a total of 6 positions match). Typically, the comparison is performed when the two sequences are aligned to produce maximum identity. Such an alignment can be achieved by using, for example, the method of Needleman et al. (1970), J. Mol. Biol. 48:443-453, which can be conveniently performed by a computer program such as the Align program (DNAstar, Inc.). The percent identity between two amino acid sequences can also be determined using the algorithm of E. Meyers and W. Miller (Comput. Appl Biosci., 4:11-17 (1988)), which has been incorporated into the ALIGN program (version 2.0), using the PAM120 weight residue table, a gap length penalty of 12, and a gap penalty of 4. In addition, the percent identity between two amino acid sequences can be determined using the algorithm of Needleman and Wunsch (J Mol Biol. 48:444-453 (1970)), which has been integrated into the GAP program of the GCG software package (available at www.gcg.com), using a Blossum 62 matrix or a PAM250 matrix, a gap weight of 16, 14, 12, 10, 8, 6 or 4, and a length weight of 1, 2, 3, 4, 5 or 6.

As used herein, the term “conservative substitution” refers to an amino acid substitution that does not adversely affect or change the expected properties of the protein/polypeptide comprising the amino acid sequence. For example, a conservative substitution can be introduced by standard techniques known in the art, such as site-directed mutagenesis and PCR-mediated mutagenesis. Conservative amino acid substitutions include a substitution of amino acid residue with an amino acid residue having similar side chain, such as substitution with a residue that is physically or functionally similar to the corresponding amino acid residue (e.g., having similar size, shape, charge, chemical properties, including the ability to form covalent bond or hydrogen bond, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Therefore, it is preferred to replace the corresponding amino acid residue with another amino acid residue from the same side chain family. Methods for identifying conservative amino acid substitutions are well known in the art (see, e.g., Brummell et al., Biochem. 32:1180-1187 (1993); Kobayashi et al. Protein Eng. 12 (10): 879-884 (1999); and Burks et al. Proc. Natl Acad. Set USA 94:412-417 (1997), which are incorporated herein by reference).

As used herein, the term “vector” refers to a nucleic acid delivery vehicle into which a polynucleotide can be inserted. When a vector is capable of expressing a protein encoded by the inserted polynucleotide, the vector is called an expression vector. The vector can be introduced into a host cell by transformation, transduction or transfection so that the genetic material elements it carries are expressed in the host cell. Vectors are well known to those skilled in the art, including but not limited to: plasmids; phagemids; cosmids; artificial chromosomes, such as yeast artificial chromosomes (YAC), bacterial artificial chromosomes (BAC) or P1-derived artificial chromosomes (PAC); bacteriophages such as λ phage or M13 phage, and animal viruses, etc. A vector may contain a variety of elements for controlling expression, including but not limited to promoter sequence, transcription start sequence, enhancer sequence, selection element and reporter gene. In addition, the vector may also contain an origin of replication.

As used herein, the term “host cell” refers to a cell that can be used to introduce a vector, including but not limited to prokaryotic cell such as Escherichia coli or Bacillus subtilis, fungal cell such as yeast cell or Aspergillus, insect cell such as S2 Drosophila cell or Sf9, or animal cell such as fibroblast, CHO cell, COS cell, NSO cell, HeLa cell, BHK cell, HEK 293 cell or human cell. In certain embodiments, the host cell is an Escherichia coli cell.

According to the present application, the term “Escherichia coli expression system” refers to an expression system consisting of Escherichia coli (strain) and a vector, wherein the Escherichia coli (strain) is derived from a commercially available strain, such as, but not limited to: ER2566, BL21 (DE3), B834 (DE3), BLR (DE3).

Those skilled in the art will understand that the design of expression vector may depend on factors such as the selection of host cell to be transformed, the desired expression level, etc. A vector can be introduced into a host cell to thereby produce a transcript, protein, or peptide, including a protein, an isolated nucleic acid molecule, etc., as described herein.

According to the present application, the term “pharmaceutically acceptable carrier and/or excipient” refers to a carrier and/or excipient that is pharmacologically and/or physiologically compatible with a subject and an active ingredient, which is well known in the art (see, for example, Remington's Pharmaceutical Sciences. Edited by Gennaro AR, 19th ed. Pennsylvania: Mack Publishing Company, 1995), and includes but is not limited to: pH regulator, surfactant, adjuvant, ionic strength enhancer. For example, the pH regulator includes but is not limited to phosphate buffer; the surfactant includes but is not limited to cationic, anionic or nonionic surfactant, such as Tween-80; the adjuvant includes but is not limited to risedronate adjuvant (e.g., zinc-aluminum hybrid adjuvant containing risedronate sodium), aluminum adjuvant (e.g., aluminum hydroxide adjuvant, aluminum phosphate adjuvant), zinc-aluminum hybrid adjuvant (e.g., FH002C), Freund's adjuvant, oil emulsion adjuvant, cytokine, TLR agonist, CpG adjuvant, liposome, AS01_{B} adjuvant, zoledronate sodium, monophosphoryl lipid A (MPL), cholesterol-containing liposome or combination thereof; the ionic strength enhancer includes but is not limited to sodium chloride.

According to the present application, the term “adjuvant” refers to a nonspecific immunoenhancer that can enhance the body's immune response to the antigen or change the type of immune response when it is delivered together with the antigen or in advance into the body. There are many kinds of adjuvants, including but not limited to risedronate adjuvant (e.g., zinc-aluminum hybrid adjuvant containing risedronate sodium), aluminum adjuvant (e.g., aluminum hydroxide adjuvant, aluminum phosphate adjuvant), zinc-aluminum hybrid adjuvant (e.g., FH002C), Freund's adjuvant, oil emulsion adjuvant, cytokine, TLR agonist, CpG adjuvant, liposome, AS01_{B} adjuvant, zoledronic acid sodium, monophosphoryl lipid A (MPL), cholesterol-containing liposome or a combination thereof. In the present application, it is particularly preferred that the adjuvant is risedronate adjuvant (e.g., zinc-aluminum hybrid adjuvant containing risedronate sodium), aluminum adjuvant (e.g., aluminum hydroxide adjuvant, aluminum phosphate adjuvant), AS01_{B} adjuvant or combination thereof.

According to the present application, the term “effective amount” refers to an amount that can effectively achieve the intended purpose. For example, an effective amount for preventing or treating a disease (e.g., VZV infection) refers to an amount that can effectively prevent, stop or delay the occurrence of a disease (e.g., VZV infection), or alleviate, reduce or treat the severity of an existing disease (e.g., a disease caused by VZV infection). Determining such an effective amount is within the capabilities of a person skilled in the art. For example, an effective amount for therapeutic use will depend on the severity of the disease to be treated, the overall state of the patient's own immune system, the patient's general condition such as age, weight and gender, the mode of administration of the drug, and other treatments administered at the same time, etc.

In the present application, the terms “polypeptide” and “protein” have the same meaning and can be used interchangeably. And in the present application, amino acids are generally represented by single-letter and three-letter abbreviations known in the art. For example, alanine can be represented by A or Ala.

As used herein, “subject” refers to an animal, such as a vertebrate. Preferably, the subject is a mammal, such as a human, bovine, equine, feline, canine, rodent or primate. Particularly preferably, the subject is a human. In the present application, the term can be used interchangeably with “patient”.

### Beneficial Effects of the Application

The truncated VZV gE protein of the present application can be expressed in Escherichia coli with a greater degree of solubility than the complete extracellular region of gE protein while retaining most of the immune epitopes, and is easy to purify without the need for fusion with GST tags, His tags or other tags that aid in protein purification. The purification method adopted by the truncated VZV gE protein of the present application does not require the use of expensive enzymes and is low in cost, the target protein does not undergo a severe denaturation-renaturation process during the purification process, resulting in minimal loss and stable protein conformation, making it suitable for large-scale industrial production.

The gE (31-358) protein provided by the present application, when used as an antigen to immunize mice without using oily stimulants, can induce a high titer of neutralizing antibodies in serum and specific cellular immune responses, which can be used to prevent and/or treat a primary infection or recurrent infection of VZV.

In addition, the truncated VZV gE protein of the present application has good reactivity with a variety of gE protein-specific monoclonal antibodies, and can be used as a detection antigen for diagnostic reagents, showing its application prospects and value in VZV diagnosis.

The embodiments of the present application will be described in detail below in conjunction with the drawings and examples, but those skilled in the art will understand that the following drawings and examples are only used to illustrate the present application, rather than to limit the scope of the present application. According to the following detailed description of the drawings and preferred embodiments, the various objects and advantages of the present application will become apparent to those skilled in the art.

### BRIEF DESCRIPTION OF THE DRAWINGS

FIG. 1 shows the results of SDS polyacrylamide gel electrophoresis for the expression of gE proteins with different truncation lengths in Escherichia coli at an induction temperature of 37° C. in Example 2 of the present application, wherein, M: molecular weight marker; S: supernatant sample of ultrasonic treatment; 2M: protein sample dissolved in 2M urea after ultrasonic precipitation and purification of inclusion bodies; 4M: protein sample dissolved in 4M urea after ultrasonic precipitation and purification of inclusion bodies; 8M: protein sample dissolved in 8M urea after ultrasonic precipitation and purification of inclusion bodies.

FIG. 2 shows the results of SDS polyacrylamide gel electrophoresis of the following samples in Example 2 of the present application: (i) the lysis supernatants of Escherichia coli expressing gE proteins with different truncation lengths at an induction temperature of 24° C., (ii) the lysis supernatants of Escherichia coli expressing gE (337-509) at different induction temperatures (16° C., 20° C., 24° C., 37° C.), and (iii) the protein sample of the lysis precipitate of Escherichia coli expressing gE (182-358) dissolved by TB8.0 and heated for solubilization. Therein, M: molecular weight marker, and the red arrow indicates the truncated gE protein band.

FIG. 3 shows the results of SDS polyacrylamide gel electrophoresis after Q-FF ion chromatography of the Escherichia coli lysate supernatants in Example 2 of the present application, which were obtained by ultrasonically lysing the Escherichia coli expressing gE proteins of different truncation lengths at an induction temperature of 24° C., and the ultrasonic lysis was performed with TB8.0. Therein, M: molecular weight marker; ↑: supernatant sample after bacterial lysis; CT: penetration sample during Q-FF ion chromatography; 0.2M: elution sample at a NaCl concentration of 200 mmol/L during Q-FF ion chromatography; 0.4M: elution sample at a NaCl concentration of 400 mmol/L during Q-FF ion chromatography; 2M: elution sample at a NaCl concentration of 2000 mmol/L during Q-FF ion chromatography; \: bacterial lysis precipitate. The red arrow indicates the truncated gE protein band.

FIG. 4 shows the SDS polyacrylamide gel electrophoresis results of various VZV gE truncated proteins at different purification stages in Example 2 of the present application, in which panel A shows the electrophoresis results of various gE (31-358) samples, wherein M: molecular weight marker; Lane 1: elution sample at a NaCl concentration of 400 mmol/L during Q-FF ion chromatography; Lane 2: penetration sample during CHT chromatography with equilibrium solution; Lane 3: elution sample at a NaCl concentration of 500 mmol/L during chromatography on Butyl column; panel B shows the electrophoresis results of various gE (31-320) samples, wherein M: molecular weight marker; Lane 1: elution sample at a NaCl concentration of 400 mmol/L during Q-FF ion chromatography; Lane 2: penetration sample during CHT chromatography with equilibrium solution; Lane 3: elution sample at a NaCl concentration of 1000 mmol/L during chromatography on Butyl column; panel C shows the electrophoresis results of various gE (128-358) samples, wherein M: molecular weight marker; Lane 1: elution sample at a NaCl concentration of 400 mmol/L during Q-FF ion chromatography; Lane 2: penetration sample during CHT chromatography with equilibrium solution; Lane 3: elution sample at a NaCl concentration of 500 mmol/L during chromatography on Butyl column.

FIG. 5 shows the analysis results of high performance gel filtration chromatography (panel A) and protein sedimentation coefficient (panel B), as well as the immune reaction results with gE neutralizing antibodies (11B11, 4A2, 11B12, 6H6, 10H6, 1B11, 4G4, 14G1, 11E3) (panel C) of the purified VZV gE (31-358) protein in Example 3.

FIG. 6 shows the binding antibody titers of mouse serum at different stages after the mice were immunized with the purified VZV gE (31-358) protein in Example 4. Panel A shows the results of Balb/C mice immunized at 0/2/4 weeks with either risedronate adjuvant or aluminum adjuvant and with immunization doses of 5 μg and 1 ug. Panel B shows the results of C57 mice immunized at 0/4 weeks with either risedronate adjuvant or AS01_{B} adjuvant and with an immunization dose of 5 ug.

FIG. 7 shows the serum neutralizing antibody titers of C57 mice immunized with purified VZV gE (31-358) protein in Example 4, in which the experimental scheme was to use either risedronate adjuvant or AS01_{B} adjuvant, the immunization dose was 5 ug, and booster immunization was given at 4 weeks after the primary immunization.

FIG. 8 shows the cytokine secretion results in C57 mice immunized with the purified VZV gE (31-358) protein in Example 4 at day 14 after single injection (panel A) and at day 30 after two injections (panel B), in which the immunization dose was 5 ug, and booster immunization was given at week 4 after the primary immunization.

### SEQUENCE INFORMATION

The description of sequences involved in the present application is provided in the below table.

TABLE 1
Sequence information

| SEQ ID NO: | Sequence and description |
|---|---|
| 1 | gE(31-303) amino acid sequence |
| | SVLRYDDFHIDEDKLDTNSVYEPYYHSDHAESSWVNRGESSRKAYDHN |
| | SPYIWPRNDYDGFLENAHEHHGVYNQGRGIDSGERLMQPTQMSAQEDL |
| | GDDTGIHVIPTLNGDDRHKIVNVDQRQYGDVFKGDLNPKPQGQRLIEV |
| | SVEENHPFTLRAPIQRIYGVRYTETWSFLPSLTCTGDAAPAIQHICLK |
| | HTTCFQDVVVDVDCAENTKEDQLAEISYRFQGKKEADQPWIVVNTSTL |
| | FDELELDPPEIEPGVLKVLRTEKQYLGVYIWNM |
| 2 | gE(31-320) amino acid sequence |
| | SVLRYDDFHIDEDKLDTNSVYEPYYHSDHAESSWVNRGESSRKAYDHN |
| | SPYIWPRNDYDGFLENAHEHHGVYNQGRGIDSGERLMQPTQMSAQEDL |
| | GDDTGIHVIPTLNGDDRHKIVNVDQRQYGDVFKGDLNPKPQGQRLIEV |
| | SVEENHPFTLRAPIQRIYGVRYTETWSFLPSLTCTGDAAPAIQHICLK |
| | HTTCFQDVVVDVDCAENTKEDQLAEISYRFQGKKEADQPWIVVNTSTL |
| | FDELELDPPEIEPGVLKVLRTEKQYLGVYIWNMRGSDGTSTYATFLVT |
| | WK |
| 3 | gE(31-358) amino acid sequence |
| | SVLRYDDFHIDEDKLDTNSVYEPYYHSDHAESSWVNRGESSRKAYDHN |
| | SPYIWPRNDYDGFLENAHEHHGVYNQGRGIDSGERLMQPTQMSAQEDL |
| | GDDTGIHVIPTLNGDDRHKIVNVDQRQYGDVFKGDLNPKPQGQRLIEV |
| | SVEENHPFTLRAPIQRIYGVRYTETWSFLPSLTCTGDAAPAIQHICLK |
| | HTTCFQDVVVDVDCAENTKEDQLAEISYRFQGKKEADQPWIVVNTSTL |
| | FDELELDPPEIEPGVLKVLRTEKQYLGVYIWNMRGSDGTSTYATFLVT |
| | WKGDEKTRNPTPAVTPQPRGAEFHMWNYHSHVFSVGDTFS |
| 4 | gE(81-358) amino acid sequence |
| | YIWPRNDYDGFLENAHEHHGVYNQGRGIDSGERLMQPTQMSAQEDLGD |
| | DTGIHVIPTLNGDDRHKIVNVDQRQYGDVFKGDLNPKPQGQRLIEVSV |
| | EENHPFTLRAPIQRIYGVRYTETWSFLPSLTCTGDAAPAIQHICLKHT |
| | TCFQDVVVDVDCAENTKEDQLAEISYRFQGKKEADQPWIVVNTSTLFD |
| | ELELDPPEIEPGVLKVLRTEKQYLGVYIWNMRGSDGTSTYATFLVTWK |
| | GDEKTRNPTPAVTPQPRGAEFHMWNYHSHVFSVGDTFS |
| 5 | gE(128-358) amino acid sequence |
| | DDTGIHVIPTLNGDDRHKIVNVDQRQYGDVFKGDLNPKPQGQRLIEVS |
| | VEENHPFTLRAPIQRIYGVRYTETWSFLPSLTCTGDAAPAIQHICLKH |
| | TTCFQDVVVDVDCAENTKEDQLAEISYRFQGKKEADQPWIVVNTSTLF |
| | DELELDPPEIEPGVLKVLRTEKQYLGVYIWNMRGSDGTSTYATFLVTW |
| | KGDEKTRNPTPAVTPQPRGAEFHMWNYHSHVFSVGDTFS |
| 6 | gE(140-330) amino acid sequence |
| | GDDRHKIVNVDQRQYGDVFKGDLNPKPQGQRLIEVSVEENHPFTLRAP |
| | IQRIYGVRYTETWSFLPSLTCTGDAAPAIQHICLKHTTCFQDVVVDVD |
| | CAENTKEDQLAEISYRFQGKKEADQPWIVVNTSTLFDELELDPPEIEP |
| | GVLKVLRTEKQYLGVYIWNMRGSDGTSTYATFLVTWKGDEKTRNPTP |
| 7 | gE(31-303) encoding nucleotide sequence |
| | TCCGTCTTGCGATACGATGATTTTCACATCGATGAAGACAAACTGGAT |
| | ACAAACTCCGTATATGAGCCTTACTACCATTCAGATCATGCGGAGTCT |
| | TCATGGGTAAATCGGGGAGAGTCTTCGCGAAAAGCGTACGATCATAAC |
| | TCACCTTATATATGGCCACGTAATGATTATGATGGATTTTTAGAGAAC |
| | GCACACGAACACCATGGGGTGTATAATCAGGGCCGTGGTATCGATAGC |
| | GGGGAACGGTTAATGCAACCCACACAAATGTCTGCACAGGAGGATCTT |
| | GGGGACGATACGGGCATCCACGTTATCCCTACGTTAAACGGCGATGAC |
| | AGACATAAAATTGTAAATGTGGACCAACGTCAATACGGTGACGTGTTT |
| | AAAGGAGATCTTAATCCAAAACCCCAAGGCCAAAGACTCATTGAGGTG |
| | TCAGTGGAAGAAAATCACCCGTTTACTTTACGCGCACCGATTCAGCGG |
| | ATTTATGGAGTCCGGTACACCGAGACTTGGAGCTTTTTGCCGTCATTA |
| | ACCTGTACGGGAGACGCAGCGCCCGCCATCCAGCATATATGTTTAAAA |
| | CATACAACATGCTTTCAAGACGTGGTGGTGGATGTGGATTGCGCGGAA |
| | AATACTAAAGAGGATCAGTTGGCCGAAATCAGTTACCGTTTTCAAGGT |
| | AAGAAGGAAGCGGACCAACCGTGGATTGTTGTAAACACGAGCACACTG |
| | TTTGATGAACTCGAATTAGACCCCCCCGAGATTGAACCGGGTGTCTTG |
| | AAAGTACTTCGGACAGAAAAACAATACTTGGGTGTGTACATTTGGAAC |
| | ATG |
| 8 | gE(31-320) encoding nucleotide sequence |
| | TCCGTCTTGCGATACGATGATTTTCACATCGATGAAGACAAACTGGAT |
| | ACAAACTCCGTATATGAGCCTTACTACCATTCAGATCATGCGGAGTCT |
| | TCATGGGTAAATCGGGGAGAGTCTTCGCGAAAAGCGTACGATCATAAC |
| | TCACCTTATATATGGCCACGTAATGATTATGATGGATTTTTAGAGAAC |
| | GCACACGAACACCATGGGGTGTATAATCAGGGCCGTGGTATCGATAGC |
| | GGGGAACGGTTAATGCAACCCACACAAATGTCTGCACAGGAGGATCTT |
| | GGGGACGATACGGGCATCCACGTTATCCCTACGTTAAACGGCGATGAC |
| | AGACATAAAATTGTAAATGTGGACCAACGTCAATACGGTGACGTGTTT |
| | AAAGGAGATCTTAATCCAAAACCCCAAGGCCAAAGACTCATTGAGGTG |
| | TCAGTGGAAGAAAATCACCCGTTTACTTTACGCGCACCGATTCAGCGG |
| | ATTTATGGAGTCCGGTACACCGAGACTTGGAGCTTTTTGCCGTCATTA |
| | ACCTGTACGGGAGACGCAGCGCCCGCCATCCAGCATATATGTTTAAAA |
| | CATACAACATGCTTTCAAGACGTGGTGGTGGATGTGGATTGCGCGGAA |
| | AATACTAAAGAGGATCAGTTGGCCGAAATCAGTTACCGTTTTCAAGGT |
| | AAGAAGGAAGCGGACCAACCGTGGATTGTTGTAAACACGAGCACACTG |
| | TTTGATGAACTCGAATTAGACCCCCCCGAGATTGAACCGGGTGTCTTG |
| | AAAGTACTTCGGACAGAAAAACAATACTTGGGTGTGTACATTTGGAAC |
| | ATGCGCGGCTCCGATGGTACGTCTACCTACGCCACGTTTTTGGTCACC |
| | TGGAAA |
| 9 | gE(31-358) encoding nucleotide sequence |
| | TCCGTCTTGCGATACGATGATTTTCACATCGATGAAGACAAACTGGAT |
| | ACAAACTCCGTATATGAGCCTTACTACCATTCAGATCATGCGGAGTCT |
| | TCATGGGTAAATCGGGGAGAGTCTTCGCGAAAAGCGTACGATCATAAC |
| | TCACCTTATATATGGCCACGTAATGATTATGATGGATTTTTAGAGAAC |
| | GCACACGAACACCATGGGGTGTATAATCAGGGCCGTGGTATCGATAGC |
| | GGGGAACGGTTAATGCAACCCACACAAATGTCTGCACAGGAGGATCTT |
| | GGGGACGATACGGGCATCCACGTTATCCCTACGTTAAACGGCGATGAC |
| | AGACATAAAATTGTAAATGTGGACCAACGTCAATACGGTGACGTGTTT |
| | AAAGGAGATCTTAATCCAAAACCCCAAGGCCAAAGACTCATTGAGGTG |
| | TCAGTGGAAGAAAATCACCCGTTTACTTTACGCGCACCGATTCAGCGG |
| | ATTTATGGAGTCCGGTACACCGAGACTTGGAGCTTTTTGCCGTCATTA |
| | ACCTGTACGGGAGACGCAGCGCCCGCCATCCAGCATATATGTTTAAAA |
| | CATACAACATGCTTTCAAGACGTGGTGGTGGATGTGGATTGCGCGGAA |
| | AATACTAAAGAGGATCAGTTGGCCGAAATCAGTTACCGTTTTCAAGGT |
| | AAGAAGGAAGCGGACCAACCGTGGATTGTTGTAAACACGAGCACACTG |
| | TTTGATGAACTCGAATTAGACCCCCCCGAGATTGAACCGGGTGTCTTG |
| | AAAGTACTTCGGACAGAAAAACAATACTTGGGTGTGTACATTTGGAAC |
| | ATGCGCGGCTCCGATGGTACGTCTACCTACGCCACGTTTTTGGTCACC |
| | TGGAAAGGGGATGAAAAAACAAGAAACCCTACGCCCGCAGTAACTCCT |
| | CAACCAAGAGGGGCTGAGTTTCATATGTGGAATTACCACTCGCATGTA |
| | TTTTCAGTTGGTGATACGTTTAGC |
| 10 | gE(81-358) encoding nucleotide sequence |
| | TATATATGGCCACGTAATGATTATGATGGATTTTTAGAGAACGCACAC |
| | GAACACCATGGGGTGTATAATCAGGGCCGTGGTATCGATAGCGGGGAA |
| | CGGTTAATGCAACCCACACAAATGTCTGCACAGGAGGATCTTGGGGAC |
| | GATACGGGCATCCACGTTATCCCTACGTTAAACGGCGATGACAGACAT |
| | AAAATTGTAAATGTGGACCAACGTCAATACGGTGACGTGTTTAAAGGA |
| | GATCTTAATCCAAAACCCCAAGGCCAAAGACTCATTGAGGTGTCAGTG |
| | GAAGAAAATCACCCGTTTACTTTACGCGCACCGATTCAGCGGATTTAT |
| | GGAGTCCGGTACACCGAGACTTGGAGCTTTTTGCCGTCATTAACCTGT |
| | ACGGGAGACGCAGCGCCCGCCATCCAGCATATATGTTTAAAACATACA |
| | ACATGCTTTCAAGACGTGGTGGTGGATGTGGATTGCGCGGAAAATACT |
| | AAAGAGGATCAGTTGGCCGAAATCAGTTACCGTTTTCAAGGTAAGAAG |
| | GAAGCGGACCAACCGTGGATTGTTGTAAACACGAGCACACTGTTTGAT |
| | GAACTCGAATTAGACCCCCCCGAGATTGAACCGGGTGTCTTGAAAGTA |
| | CTTCGGACAGAAAAACAATACTTGGGTGTGTACATTTGGAACATGCGC |
| | GGCTCCGATGGTACGTCTACCTACGCCACGTTTTTGGTCACCTGGAAA |
| | GGGGATGAAAAAACAAGAAACCCTACGCCCGCAGTAACTCCTCAACCA |
| | AGAGGGGCTGAGTTTCATATGTGGAATTACCACTCGCATGTATTTTCA |
| | GTTGGTGATACGTTTAGC |
| 11 | gE(128-358) encoding nucleotide sequence |
| | GACGATACGGGCATCCACGTTATCCCTACGTTAAACGGCGATGACAGA |
| | CATAAAATTGTAAATGTGGACCAACGTCAATACGGTGACGTGTTTAAA |
| | GGAGATCTTAATCCAAAACCCCAAGGCCAAAGACTCATTGAGGTGTCA |
| | GTGGAAGAAAATCACCCGTTTACTTTACGCGCACCGATTCAGCGGATT |
| | TATGGAGTCCGGTACACCGAGACTTGGAGCTTTTTGCCGTCATTAACC |
| | TGTACGGGAGACGCAGCGCCCGCCATCCAGCATATATGTTTAAAACAT |
| | ACAACATGCTTTCAAGACGTGGTGGTGGATGTGGATTGCGCGGAAAAT |
| | ACTAAAGAGGATCAGTTGGCCGAAATCAGTTACCGTTTTCAAGGTAAG |
| | AAGGAAGCGGACCAACCGTGGATTGTTGTAAACACGAGCACACTGTTT |
| | GATGAACTCGAATTAGACCCCCCCGAGATTGAACCGGGTGTCTTGAAA |
| | GTACTTCGGACAGAAAAACAATACTTGGGTGTGTACATTTGGAACATG |
| | CGCGGCTCCGATGGTACGTCTACCTACGCCACGTTTTTGGTCACCTGG |
| | AAAGGGGATGAAAAAACAAGAAACCCTACGCCCGCAGTAACTCCTCAA |
| | CCAAGAGGGGCTGAGTTTCATATGTGGAATTACCACTCGCATGTATTT |
| | TCAGTTGGTGATACGTTTAGC |
| 12 | gE(140-330) encoding nucleotide sequence |
| | GGCGATGACAGACATAAAATTGTAAATGTGGACCAACGTCAATACGGT |
| | GACGTGTTTAAAGGAGATCTTAATCCAAAACCCCAAGGCCAAAGACTC |
| | ATTGAGGTGTCAGTGGAAGAAAATCACCCGTTTACTTTACGCGCACCG |
| | ATTCAGCGGATTTATGGAGTCCGGTACACCGAGACTTGGAGCTTTTTG |
| | CCGTCATTAACCTGTACGGGAGACGCAGCGCCCGCCATCCAGCATATA |
| | TGTTTAAAACATACAACATGCTTTCAAGACGTGGTGGTGGATGTGGAT |
| | TGCGCGGAAAATACTAAAGAGGATCAGTTGGCCGAAATCAGTTACCGT |
| | TTTCAAGGTAAGAAGGAAGCGGACCAACCGTGGATTGTTGTAAACACG |
| | AGCACACTGTTTGATGAACTCGAATTAGACCCCCCCGAGATTGAACCG |
| | GGTGTCTTGAAAGTACTTCGGACAGAAAAACAATACTTGGGTGTGTAC |
| | ATTTGGAACATGCGCGGCTCCGATGGTACGTCTACCTACGCCACGTTT |
| | TTGGTCACCTGGAAAGGGGATGAAAAAACAAGAAACCCTACGCCC |
| 13 | Primer gE-1-F nucleotide sequence |
| | GGGACAGTTAATAAACCTGT |
| 14 | Primer gE-546-R nucleotide sequence |
| | TGCAAGCCCTCCGGTCCATG |
| 15 | Primer B11-gE-31-F nucleotide sequence |
| | AAGAAGGAGATATACATATGTCCGTCTTGCGATACGATGA |
| 16 | Primer B11-gE-358-R nucleotide sequence |
| | TTGTTAGCAGCCGGATCTCAGCTAAACGTATCACCAACTG |
| 17 | Primer B11-NdeI-VF nucleotide sequence |
| | CATATGTATATCTCCTTCTT |
| 18 | Primer B11-TGA-VR nucleotide sequence |
| | TGAGATCCGGCTGCTAACAA |
| 19 | amino acid sequence of gE in full-length |
| | MGTVNKPVVGVLMGFGIITGTLRITNPVRASVLRYDDFHIDEDKLDTN |
| | SVYEPYYHSDHAESSWVNRGESSRKAYDHNSPYIWPRNDYDGFLENAH |
| | EHHGVYNQGRGIDSGERLMQPTQMSAQEDLGDDTGIHVIPTLNGDDRH |
| | KIVNVDQRQYGDVFKGDLNPKPQGQRLIEVSVEENHPFTLRAPIQRIY |
| | GVRYTETWSFLPSLTCTGDAAPAIQHICLKHTTCFQDVVVDVDCAENT |
| | KEDQLAEISYRFQGKKEADQPWIVVNTSTLFDELELDPPEIEPGVLKV |
| | LRTEKQYLGVYIWNMRGSDGTSTYATFLVTWKGDEKTRNPTPAVTPQP |
| | RGAEFHMWNYHSHVFSVGDTFSLAMHLQYKIHEAPFDLLLEWLYVPID |
| | PTCQPMRLYSTCLYHPNAPQCLSHMNSGCTFTSPHLAQRVASTVYQNC |
| | EHADNYTAYCLGISHMEPSFGLILHDGGTTLKFVDTPESLSGLYVFVV |
| | YFNGHVEAVAYTVVSTVDHFVNAIEERGFPPTAGQPPATTKPKEITPV |
| | NPGTSPLLRYAAWTGGLAAVVLLCLVIFLICTAKRMRVKAYRVDKSPY |
| | NQSMYYAGLPVDDFEDSESTDTEEEFGNAIGGSHGGSSYTVYIDKTR |
| 20 | gE(31-330) amino acid sequence |
| | SVLRYDDFHIDEDKLDTNSVYEPYYHSDHAESSWVNRGESSRKAYDHN |
| | SPYIWPRNDYDGFLENAHEHHGVYNQGRGIDSGERLMQPTQMSAQEDL |
| | GDDTGIHVIPTLNGDDRHKIVNVDQRQYGDVFKGDLNPKPQGQRLIEV |
| | SVEENHPFTLRAPIQRIYGVRYTETWSFLPSLTCTGDAAPAIQHICLK |
| | HTTCFQDVVVDVDCAENTKEDQLAEISYRFQGKKEADQPWIVVNTSTL |
| | FDELELDPPEIEPGVLKVLRTEKQYLGVYIWNMRGSDGTSTYATFLVT |
| | WKGDEKTRNPTP |
| 21 | gE(74-303) amino acid sequence |
| | AYDHNSPYIWPRNDYDGFLENAHEHHGVYNQGRGIDSGERLMQPTQMS |
| | AQEDLGDDTGIHVIPTLNGDDRHKIVNVDQRQYGDVFKGDLNPKPQGQ |
| | RLIEVSVEENHPFTLRAPIQRIYGVRYTETWSFLPSLTCTGDAAPAIQ |
| | HICLKHTTCFQDVVVDVDCAENTKEDQLAEISYRFQGKKEADQPWIVV |
| | NTSTLFDELELDPPEIEPGVLKVLRTEKQYLGVYIWNM |
| 22 | gE(74-320) amino acid sequence |
| | AYDHNSPYIWPRNDYDGFLENAHEHHGVYNQGRGIDSGERLMQPTQMS |
| | AQEDLGDDTGIHVIPTLNGDDRHKIVNVDQRQYGDVFKGDLNPKPQGQ |
| | RLIEVSVEENHPFTLRAPIQRIYGVRYTETWSFLPSLTCTGDAAPAIQ |
| | HICLKHTTCFQDVVVDVDCAENTKEDQLAEISYRFQGKKEADQPWIVV |
| | NTSTLFDELELDPPEIEPGVLKVLRTEKQYLGVYIWNMRGSDGTSTYA |
| | TFLVTWK |
| 23 | gE(74-330) amino acid sequence |
| | AYDHNSPYIWPRNDYDGFLENAHEHHGVYNQGRGIDSGERLMQPTQMS |
| | AQEDLGDDTGIHVIPTLNGDDRHKIVNVDQRQYGDVFKGDLNPKPQGQ |
| | RLIEVSVEENHPFTLRAPIQRIYGVRYTETWSFLPSLTCTGDAAPAIQ |
| | HICLKHTTCFQDVVVDVDCAENTKEDQLAEISYRFQGKKEADQPWIVV |
| | NTSTLFDELELDPPEIEPGVLKVLRTEKQYLGVYIWNMRGSDGTSTYA |
| | TFLVTWKGDEKTRNPTP |
| 24 | gE(74-358) amino acid sequence |
| | AYDHNSPYIWPRNDYDGFLENAHEHHGVYNQGRGIDSGERLMQPTQMS |
| | AQEDLGDDTGIHVIPTLNGDDRHKIVNVDQRQYGDVFKGDLNPKPQGQ |
| | RLIEVSVEENHPFTLRAPIQRIYGVRYTETWSFLPSLTCTGDAAPAIQ |
| | HICLKHTTCFQDVVVDVDCAENTKEDQLAEISYRFQGKKEADQPWIVV |
| | NTSTLFDELELDPPEIEPGVLKVLRTEKQYLGVYIWNMRGSDGTSTYA |
| | TFLVTWKGDEKTRNPTPAVTPQPRGAEFHMWNYHSHVFSVGDTFS |
| 25 | gE(81-303) amino acid sequence |
| | YIWPRNDYDGFLENAHEHHGVYNQGRGIDSGERLMQPTQMSAQEDLGD |
| | DTGIHVIPTLNGDDRHKIVNVDQRQYGDVFKGDLNPKPQGQRLIEVSV |
| | EENHPFTLRAPIQRIYGVRYTETWSFLPSLTCTGDAAPAIQHICLKHT |
| | TCFQDVVVDVDCAENTKEDQLAEISYRFQGKKEADQPWIVVNTSTLFD |
| | ELELDPPEIEPGVLKVLRTEKQYLGVYIWNM |
| 26 | gE(81-320) amino acid sequence |
| | YIWPRNDYDGFLENAHEHHGVYNQGRGIDSGERLMQPTQMSAQEDLGD |
| | DTGIHVIPTLNGDDRHKIVNVDQRQYGDVFKGDLNPKPQGQRLIEVSV |
| | EENHPFTLRAPIQRIYGVRYTETWSFLPSLTCTGDAAPAIQHICLKHT |
| | TCFQDVVVDVDCAENTKEDQLAEISYRFQGKKEADQPWIVVNTSTLFD |
| | ELELDPPEIEPGVLKVLRTEKQYLGVYIWNMRGSDGTSTYATFLVTWK |
| 27 | gE(81-330) amino acid sequence |
| | YIWPRNDYDGFLENAHEHHGVYNQGRGIDSGERLMQPTQMSAQEDLGD |
| | DTGIHVIPTLNGDDRHKIVNVDQRQYGDVFKGDLNPKPQGQRLIEVSV |
| | EENHPFTLRAPIQRIYGVRYTETWSFLPSLTCTGDAAPAIQHICLKHT |
| | TCFQDVVVDVDCAENTKEDQLAEISYRFQGKKEADQPWIVVNTSTLFD |
| | ELELDPPEIEPGVLKVLRTEKQYLGVYIWNMRGSDGTSTYATFLVTWK |
| | GDEKTRNPTP |
| 28 | gE(128-303) amino acid sequence |
| | DDTGIHVIPTLNGDDRHKIVNVDQRQYGDVFKGDLNPKPQGQRLIEVS |
| | VEENHPFTLRAPIQRIYGVRYTETWSFLPSLTCTGDAAPAIQHICLKH |
| | TTCFQDVVVDVDCAENTKEDQLAEISYRFQGKKEADQPWIVVNTSTLF |
| | DELELDPPEIEPGVLKVLRTEKQYLGVYIWNM |
| 29 | gE(128-320) amino acid sequence |
| | DDTGIHVIPTLNGDDRHKIVNVDQRQYGDVFKGDLNPKPQGQRLIEVS |
| | VEENHPFTLRAPIQRIYGVRYTETWSFLPSLTCTGDAAPAIQHICLKH |
| | TTCFQDVVVDVDCAENTKEDQLAEISYRFQGKKEADQPWIVVNTSTLF |
| | DELELDPPEIEPGVLKVLRTEKQYLGVYIWNMRGSDGTSTYATFLVTW |
| | K |
| 30 | gE(128-330) amino acid sequence |
| | DDTGIHVIPTLNGDDRHKIVNVDQRQYGDVFKGDLNPKPQGQRLIEVS |
| | VEENHPFTLRAPIQRIYGVRYTETWSFLPSLTCTGDAAPAIQHICLKH |
| | TTCFQDVVVDVDCAENTKEDQLAEISYRFQGKKEADQPWIVVNTSTLF |
| | DELELDPPEIEPGVLKVLRTEKQYLGVYIWNMRGSDGTSTYATFLVTW |
| | KGDEKTRNPTP |
| 31 | gE(Bac) amino acid sequence |
| | RITNPVRASVLRYDDFHIDEDKLDTNSVYEPYYHSDHAESSWVNRGES |
| | SRKAYDHNSPYIWPRNDYDGFLENAHEHHGVYNQGRGIDSGERLMQPT |
| | QMSAQEDLGDDTGIHVIPTLNGDDRHKIVNVDQRQYGDVFKGDLNPKP |
| | QGQRLIEVSVEENHPFTLRAPIQRIYGVRYTETWSFLPSLTCTGDAAP |
| | AIQHICLKHTTCFQDVVVDVDCAENTKEDQLAEISYRFQGKKEADQPW |
| | IVVNTSTLFDELELDPPEIEPGVLKVLRTEKQYLGVYIWNMRGSDGTS |
| | TYATFLVTWKGDEKTRNPTPAVTPQPRGAEFHMWNYHSHVFSVGDTFS |
| | LAMHLQYKIHEAPFDLLLEWLYVPIDPTCQPMRLYSTCLYHPNAPQCL |
| | SHMNSGCTFTSPHLAQRVASTVYQNCEHADNYTAYCLGISHMEPSFGL |
| | ILHDGGTTLKFVDTPESLSGLYVFVVYFNGHVEAVAYTVVSTVDHFVN |
| | AIEERGFPPTAGQPPATTKPKEITPVNPGTSPLLR |

### Specific Models for Carrying Out the Application

The present application is now described with reference to the following examples which are intended to illustrate the present application (but not to limit the present application).

Unless otherwise specified, the molecular biology experimental methods and immunoassays used in the present application were basically performed according to the methods of J. Sambrook et al., Molecular Cloning: A Laboratory Manual, 2nd Edition, Cold Spring Harbor Laboratory Press, 1989, and F. M. Ausubel et al., Molecular Biology Experiment Manual, 3rd edition, John Wiley & Sons, Inc., 1995; the restriction endonucleases were used according to the conditions recommended by the product manufacturer. Those skilled in the art will appreciate that the examples describe the present application by way of example and are not intended to limit the scope of the present application.

### Example 1: Cloning of Plasmid Containing Truncated gE Protein Encoding Nucleotide Sequence

### Preparation of a Template for Genes of VZV gE Extracellular Segment

A plasmid containing a VZV glycoprotein E (gE) encoding nucleotide sequence was synthesized using the DNA sequence of GenBank: AY253715.1 as a template, in which gE-1-F (SEQ ID NO:13) was used as the forward primer, gE-546-R (SEQ ID NO:14) was used as the reverse primer, and the PCR reaction was performed in PCR instrument (Biometra, T3) according to the following conditions:

Denaturation at 94° C. for 5 min; denaturation at 94° C. for 50 sec, annealing at 57° C. for 50 sec, extension at 72° C. for 2 min (25 cycles); extension at 72° C. for 10 min.

A specific product with a size of about 1.6 kb was obtained by amplification, and used as the template for preparing the DNA fragment encoding the truncated VZV-gE protein in the present application.

### Construction of Vector Containing a Gene of Truncated VZV-gE

Taking the construction of clone containing the nucleotide sequence encoding gE (31-358) (which amino acid sequence was shown in SEQ ID NO: 3) as an example, B11-gE-31-F (SEQ ID NO: 15) was taken as the forward primer, a restriction endonuclease NdeI site was introduced at its 5′ end, the sequence of the NdeI site was CATATG, and ATG was the start codon in the Escherichia coli system; B11-gE-358-R (SEQ ID NO: 16) was used as the reverse primer, and the 1.6 kb DNA fragment obtained in the previous step was used as the template for the second PCR reaction. The PCR reaction was carried out in a PCR thermal cycler (Biometra T3) according to the following conditions:

Denaturation at 94° C. for 5 min; denaturation at 94° C. for 50 sec, annealing at 57° C. for 50 sec, extension at 72° C. for 2 min (25 cycles); extension at 72° C. for 10 min.

A specific DNA fragment with a size of about 1.0 kb was obtained by amplification. Then the PCR product was Gibson assembled with the PCR amplified fragment of the vector pTO-T7 (Luo Wenxin et al., Journal of Biotechnology, 2000, 16:53-57), which was amplified using SEQ ID NO: 17, B11-NdeI-VF as forward primer, SEQ ID NO: 18, B11-TGA-VR as reverse primer, to obtain the positive clone pTO-T7-gE (31-358) with the truncated VZV-gE gene inserted.

Similarly, clones of other truncations were obtained.

Sangon Biotech (Shanghai) was entrusted for sequencing, and the target nucleotide sequence inserted into the pTO-T7-gE (31-358) plasmid was determined to be SEQ ID NO: 9, with T7 (+)/(−) primers used as the sequencing primers, and the amino acid sequence encoded thereby was SEQ ID NO: 3, the protein corresponding to this sequence is the VZV-gE protein with the N-terminal truncated to amino acid position 31 and the C-terminal truncated to amino acid position 358, named gE (31-358). The other truncated clones were named similarly: gE (31-303) (the amino acid sequence was shown in SEQ ID NO: 1, and the encoding nucleotide sequence was shown in SEQ ID NO: 7), gE (31-320) (the amino acid sequence was shown in SEQ ID NO: 2, and the encoding nucleotide sequence was shown in SEQ ID NO: 8), gE (81-358) (the amino acid sequence was shown in SEQ ID NO: 4, and the encoding nucleotide sequence was shown in SEQ ID NO: 10), gE (128-358) (the amino acid sequence was shown in SEQ ID NO: 5, and the encoding nucleotide sequence was shown in SEQ ID NO: 11), gE (140-330) (the amino acid sequence was shown in SEQ ID NO: 6, and the encoding nucleotide sequence was shown in SEQ ID NO: 12).

1 μL of pTO-T7-gE (31-358) plasmid (0.15 mg/ml) was taken to transform 40 μL of competent Escherichia coli ER2566 prepared by calcium chloride method (purchased from New England Biolabs), spread on a solid LB medium (ingredients: 10 g/L peptone, 5 g/L yeast powder, 10 g/L sodium chloride, the same below) with kanamycin (final concentration 25 mg/mL, the same below), and subjected to stationary culture at 37° C. for 10 to 12 hours until single colonies were clearly discernible. Single colonies were picked and placed in a test tube containing 4 mL of liquid LB medium with kanamycin, and subjected to shaking culture at 37° C. and 220 rpm for 10 hours. 1 mL of the bacterial culture was taken therefrom, and freeze-dried and stored at −70° C. Other truncation clones were stored in the same way.

### Example 2: Large-Scale Expression and Purification of Truncated gE Proteins

5 μL of the bacterial suspension of clone for each truncation prepared in Example 1 was taken out from a −70° C. ultra-low temperature refrigerator, inoculated into 5 mL of liquid LB medium containing kanamycin, subjected to shaking culture at 37° C. and 180 rpm until OD600 reached about 0.5, then transferred to 500 mL of LB medium containing kanamycin, and subjected to shaking culture at 37° C. and 180 rpm for 2 to 4 hours. Low temperature induction: when OD600 reached about 0.6, the temperature of the medium was reduced to 24° C., IPTG was added to a final concentration of 0.4 mM, and induction was performed under shaking culture at 24° C. for 10 hours; 37° C. induction: when OD600 reached about 1.5, IPTG was added to a final concentration of 0.4 mM, and induction was performed under shaking culture at 37° C. for 4 hours.

After induction, bacterial cells were collected by centrifugation at 7000 g for 5 minutes. The cells were resuspended in lysis buffer (20 mM Tris buffer, pH 8.0) with a ratio of 1 g of cells to 10 mL of lysis buffer, subjected to ice bath, and treated with an ultrasonic disruptor (Sonics VCX750 ultrasonic disruptor) (treatment conditions: working time 15 min, pulse for 2 s, pause for 4 s, output power 55%). The bacterial lysate was centrifuged at 13500 rpm (30000 g) for 15 min in a microcentrifuge with a JA-14 rotor used, and the supernatant and precipitate were separated for the next step.

### Purification of Inclusion Bodies of gE Protein Truncations

The centrifugal precipitate (i.e., inclusion bodies) of lysate induced at 37° C. conditions was washed with an equal volume of 2% Triton-100, shaken for 30 min, centrifuged and the supernatant was discarded; then the precipitate was resuspended with 20 mM Tris-HCl at pH 8.0, shaken for 30 min, centrifuged and the supernatant was discarded; then the precipitate was resuspended with 2M urea, shaken at 37° C. for 30 min, centrifuged to obtain a supernatant and a precipitate; the supernatant was retained, and the precipitate was resuspended with an equal volume of 4M urea, shaken at 37° C. for 30 min, centrifuged at 12000 rpm, 4° C. for 15 min to obtain a supernatant and a precipitate; the supernatant (i.e., the supernatant of 4M urea dissolution) was retained, and the precipitate was resuspended with an equal volume of 8M urea, shaken at 37° C. for 30 min, centrifuged, and the supernatant (i.e., the supernatant of 8M urea dissolution) was retained.

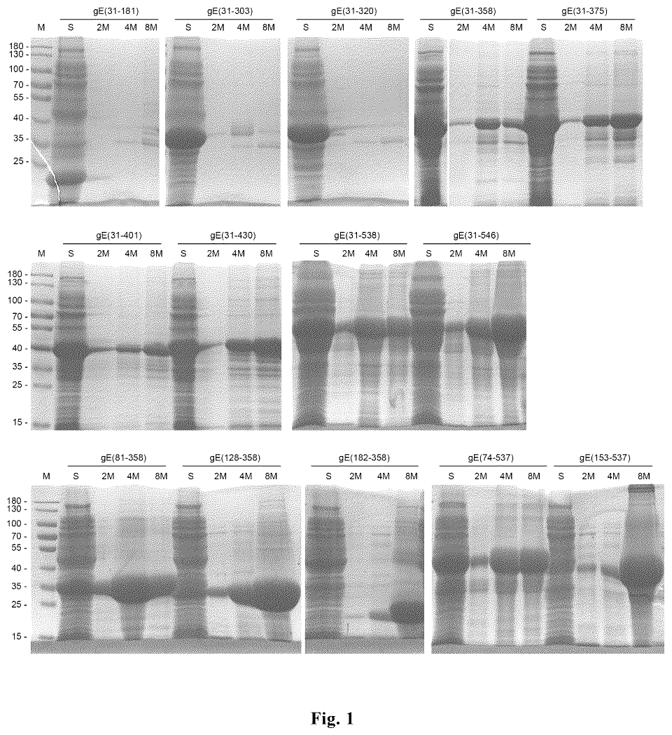

The results of SDS-PAGE analytic electrophoresis of the various fractions obtained were shown in FIG. 1. The results showed that: (1) when the N-terminal of the gE protein was fixedly truncated to amino acid position 31 and the C-terminal was sequentially truncated to amino acid positions 181/303/320/358/375/401/430/538/546, the total amount of inclusion body protein gradually increased, and the amount of inclusion body dissolved by 4M urea gradually decreased, while the amount of inclusion body dissolved by 8M urea gradually increased; 2 when the C-terminal of the gE protein was fixedly truncated to amino acid position 358 and the N-terminal was sequentially truncated to amino acid positions 81/128/182, the amount of gE protein in the ultrasonic supernatant gradually decreased, and the ratio of the amount of protein in the ultrasonic supernatant to the total amount of inclusion body protein decreased, and it was also found that the inclusion body protein gradually changed from being dissolved in 4M urea to being dissolved in 8M urea; (3) when the C-terminal of the gE protein was fixedly truncated to amino acid position 537 and the N-terminal was sequentially truncated to amino acid positions 74/153, the amount of gE protein in the ultrasonic supernatant gradually decreased, and the ratio of the amount of protein in the ultrasonic supernatant to the total amount of inclusion body protein decreased, and it was also found that the inclusion body protein gradually changed from being dissolved in 4M urea to being dissolved in 8M urea. In summary, the above results showed that under 37° C. induction, the gE protein truncated at N-terminal tended to form inclusion bodies when exogenously expressed in Escherichia coli, and the inclusion bodies required relatively higher concentration urea to dissolve; while the gE protein truncated at C-terminal tended to soluble expression when exogenously expressed in Escherichia coli, the amount of inclusion bodies formed decreased with the increase of truncation degree, and the inclusion bodies were more soluble in low concentration urea.

### Purification of gE Protein Truncation by Anion Exchange Chromatography

The supernatant sample of the lysate under 24° C. induction conditions was filtered using a 0.22 μm pore size filter membrane, and the sample was used for the next step of anion exchange chromatography;

Instrument system: AKTA explorer 100 preparative liquid chromatography system produced by GE Healthcare (formerly Amershan Pharmacia company).
Chromatographic medium: Q Sepharose 4 Fast Flow.
Column volume: 15 mm×20 cm
Buffer: 20 mM Tris buffer pH8.0;
   20 mM Tris buffer pH8.0 2M NaCl.
Flow rate: 8 mL/min
Detector wavelength: 280 nm.

The sample was the lysis supernatant of bacterial cells containing truncated gE proteins of different lengths.

The elution procedure comprised: 200 mM NaCl was used to elute impurities, 400 mM NaCl was used to elute the target protein, and the fraction eluted by 400 mM NaCl was collected.

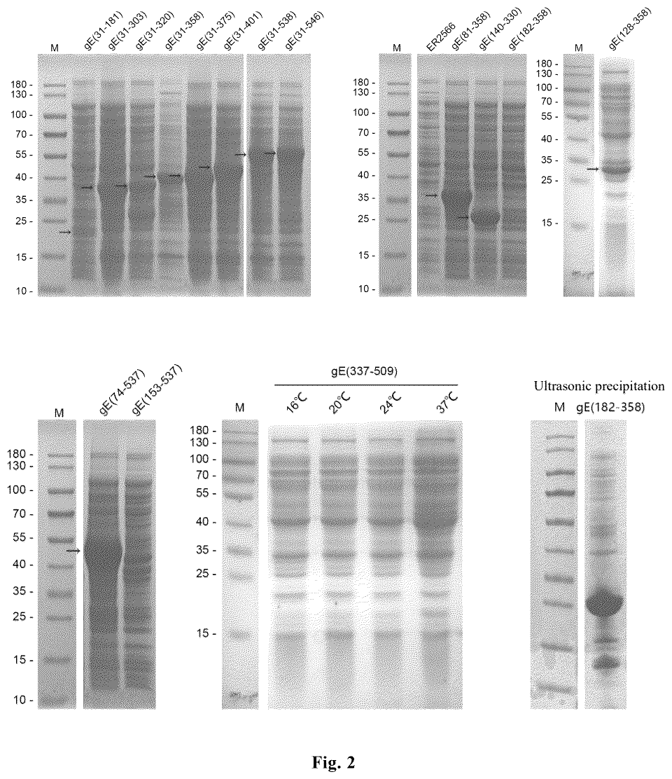

The SDS-PAGE analysis results of the various fractions obtained were shown in FIGS. 2 and 3. The results in FIG. 2 showed that similar to the expression induced at 37° C., when the C-terminal of gE protein was fixedly truncated to amino acid position 358 (330) and the N-terminal was sequentially truncated to amino acid positions 81/128/140/182, the amount of gE protein in the supernatant of ultrasonic treatment decreased gradually, and gE (182-358) was mainly expressed in the form of inclusion bodies. When the gE protein was fixedly truncated to amino acid position 537 at the C-terminal and sequentially truncated to amino acid positions 74/153 at the N-terminal, the amount of gE protein in the supernatant of ultrasonic treatment decreased gradually; and gE (337-509) protein had no obvious soluble expression at different induction temperatures. To summarize the above results, it could be seen that similar to the expression under 37° C. induction, under 24° C. induction, a certain degree of C-terminal truncation promoted the soluble expression of gE protein in Escherichia coli, while excessive N-terminal truncation caused the protein to change from soluble expression to inclusion body expression.

The results in FIG. 3 showed that C-terminal truncation had certain benefits for protein purification, enabling the protein to achieve column purification effects of low-salt binding and high-salt elution under this purification condition, which is specifically manifested in that the untruncated or slightly truncated gE extracellular segment (31-375/401/430/538/546) in the lysis supernatant of Escherichia coli could not be bound to the Q-FF column under this purification condition and almost all of it penetrated through, while the further C-terminally truncated gE protein (31-358/320/303/181) could be bound to the column and eluted at a salt concentration of 0.4 M. The gE (81-358) and gE (128-358) with truncations at both N-terminal and C-terminal could also be bound to the column and eluted at 0.4M salt concentration, the gE (74-537) with truncation at only N-terminal could not bind to the chromatography column, and the gE (153-537) and gE (182-358) with further truncation at N-terminal had no expression in supernatant. To summarize the above results, it could be seen that a certain degree of C-terminal truncation promoted the gE protein in the supernatant of ultrasonic treatment to bind to the anion column under TB8.0 buffer conditions to achieve the purification effect.

Next, taking the gE (31-358) protein as an example, the subsequent purification process of the supernatant of the lysate was described:

### CHT (Hydroxyapatite) Purification of gE (31-358)

Instrument system: AKTA explorer 100 preparative liquid chromatography system produced by GE Healthcare (formerly Amershan Pharmacia company).
Chromatographic medium: CHT-II
Column volume: 15 mm×20 cm
Buffer: 5 mM phosphate buffer pH8.0, 0.4M NaCl.
Equilibrium solution: 20 mM Tris buffer pH8.0, 0.4M NaCl.
Eluent: 200 mM phosphate buffer pH8.0, 0.4M NaCl.
Flow rate: 8 mL/min.
Detector wavelength: 280 nm.
Sample: elution product at 400 mM NaCl during Q Sepharose 4 Fast Flow chromatography.
Elution procedure: the penetration was collected after loading, and fractions were continuously collected during column equilibration with equilibrium solution after the loading was completed.

The penetration product of the equilibrium was collected.

### HIC (Hydrophobic Interaction Chromatography) Purification of gE (31-358)

Instrument system: AKTA explorer 100 preparative liquid chromatography system produced by GE Healthcare (formerly Amershan Pharmacia company).
Chromatographic medium: Butyl Sepharose 4 Fast Flow
Column volume: 15 mm×20 cm
Buffer: 20 mM Tris buffer pH8.0, 1.5M NaCl.
Eluent: 20 mM Tris buffer pH8.0
Flow rate: 8 mL/min.
Detector wavelength: 280 nm.

The sample was: CHT penetration product, which was treated by adding an appropriate amount of salt to reach a salt concentration of 1.5M.

The elution procedure comprised: 500 mM NaCl was used to elute the target protein, and 0 mM NaCl was used to elute the impurity protein.

The elution product was collected at a concentration of 500 mM NaCl to obtain a purified gE (31-358) sample.

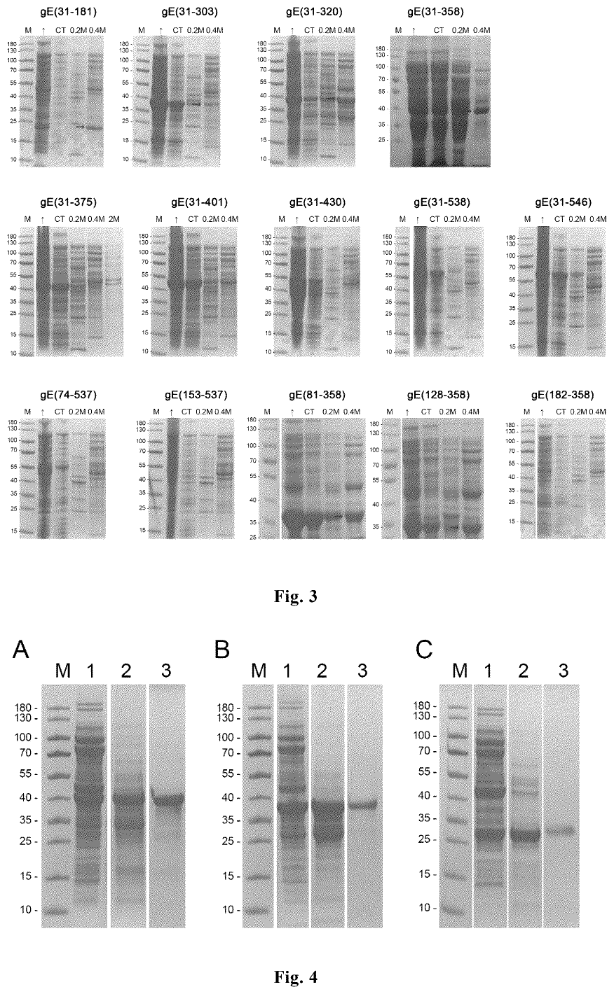

The SDS-PAGE analytic electrophoresis results of the samples of gE (31-358), gE (31-320) and gE (128-358) after purification at each stage were shown in FIG. 4. The results showed that after anion chromatography, hydroxyapatite chromatography, and hydrophobic interaction chromatography, VZV gE truncation proteins with a purity of more than 90% could be obtained.

In the following example, the gE (31-358) protein was taken as an example to detect its protein properties.

### Example 3: Analysis of Properties of gE (31-358) Protein

### Analysis of gE (31-358) Protein by High-Performance Gel Filtration Chromatography

The instrument was a Waters analytical high-performance liquid chromatograph, and a TSK Gel G5000PW column was used.

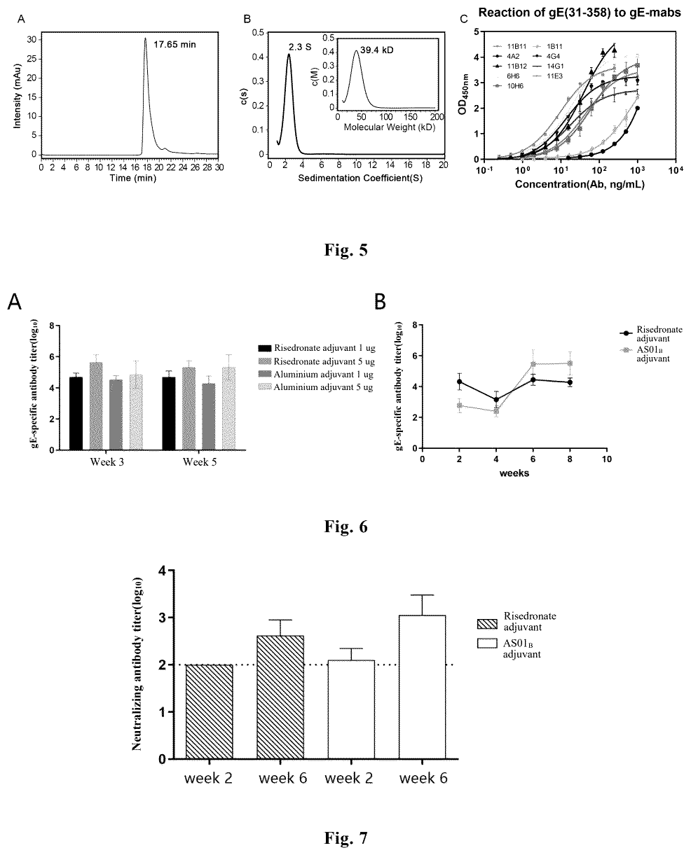

The results of high-performance gel filtration chromatography of the VZV gE (31-358) protein purified in Example 2 were shown in panel A of FIG. 5, which showed that the gE (31-358) protein obtained in Example 2 had good purity and uniformity.

### Analysis of Sedimentation Rate of gE (31-358) Protein

The instrument was a Beckman-XL-A analytical ultracentrifuge, an An60-Ti rotor was used, rotation speed was 30,000 rpm, and the collected data were fitted and analyzed using SEDFIT software.

The sedimentation coefficient analysis results of the VZV gE (31-358) protein purified in Example 2 were shown in panel B of FIG. 5. It could be seen that the sedimentation coefficient C(s) of the VZV gE (31-358) protein obtained in Example 2 was about 2.3 S, and the molecular weight was about 39.4 kDa, which was consistent with the SDS-PAGE results, and the protein purity and homogeneity were good.

### Activity Analysis of gE (31-358) Protein

The gE (31-358) protein obtained in Example 2 was coated (100 ng/well), incubated at 37° C. for 2 hours, and blocked with 1× ED at room temperature for 2 hours after washing the plate. The gE protein-specific neutralizing monoclonal antibody (screened by our laboratory through the gE protein expressed by the baculovirus expression system, see: Liu, J., Zhu, R., Ye, X., et al. (2015). A monoclonal antibody-based VZV glycoprotein E quantitative assay and its application on antigen quantitation in VZV vaccine. Applied microbiology and biotechnology, 99 (11), 4845-4853. https://doi.org/10.1007/s00253-015-6602-5) was used at a concentration of 0.1 or 1 μg/mL in the first well, and then diluted 2-fold in a series and incubated at 37° C. for 0.5 hours. The plate was washed 5 times, and the secondary antibody GAM-HRP (1:5000) was added, incubated at 37° C. for 0.5 hours, washed 5 times, and color development was performed and terminated after 10 min. The plate was detected at a wavelength of 450 nm using an ELISA reader, and GraphPad Prism 5 (GraphPad, USA) software was used for data analysis. The results were shown in panel C of FIG. 5, which showed that the gE (31-358) protein obtained in Example 2 maintained good reactivity with the gE-specific monoclonal antibody.

### Example 4: Immunogenicity Analysis of gE (31-358)

### Detection of Binding Antibody Titer Induced by gE (31-358)

The mice used in this experiment were female, 6-week-old BALB/C or C57 mice. The gE (31-358) protein prepared by the method of Example 2 was injected intramuscularly into the mice for immunization using aluminum adjuvant, risedronate adjuvant (the aluminum adjuvant and risedronate adjuvant were prepared by our laboratory, see: Wu, Y., Huang, X., Yuan, L., et al. (2021). A recombinant spike protein subunit vaccine confers protective immunity against SARS-COV-2 infection and transmission in hamsters. Science translational medicine, 13 (606), eabg1143. https://doi.org/10.1126/scitranslmed.abg1143) or AS01_{B} adjuvant (purchased from GSK), with an injection volume of 0.05 mL and a dose of 5 μg or 1 μg. The primary immunization was performed at week 0, and the booster immunization was performed at week 2 and/or week 4. ELISA was used to detect the binding antibody level in the serum after antigen immunization, in which the gE (Bac) protein (the amino acid sequence was shown in SEQ ID NO: 31) obtained by the baculovirus insect cell expression system and GAM-HRP were the capture antigen and detection antibody, respectively. The binding titer was defined as the highest serum dilution that resulted in an absorbance value greater than the critical value, and the critical value was calculated as the average value of the negative control OD450 value plus three times the standard deviation (s.d.). The detection results of binding antibody titer in immune serum were shown in FIG. 6. The results showed that mice immunized with gE (31-358) protein combined with different adjuvants could induce gE protein-specific binding antibodies in the serum; in which, the binding antibody titer increased significantly after the first immunization, and after one or two booster immunizations, the antibody titer could reach more than 104 to 105 times.

Determination of Neutralizing Antibody Titer in Serum after Immunization

The serum neutralizing antibody titer at week 2 and week 6 (i.e., two weeks after the first immunization/boosting immunization) of the above immunized mouse was detected through the serum antibody-mediated virus neutralization experiment. One day in advance, ARPE-19 cells (stored in this laboratory) were plated in a 24-well cell culture plate, and cells were used for infection when the cell density reached 70% to 80% per well. Virus protection solution (25 mM histidine, 150 mM NaCl, 9% sucrose, pH7.4) was used to prepare a virus working solution containing vOka virus (500 to 1000 pfu/mL, prepared by harvesting vOka virus passaged in ARPE-19 cells) and 10% (v/v) guinea pig serum (purchased from Beijing Bersee Science and Technology Co., Ltd., Cat. No.: BM361Y). The serum samples to be tested were inactivated at 56° C. for 30 min, and then serially diluted with the virus working solution. The diluted serum mixtures were incubated at 37° C. for 1 h. The culture medium in the 24-well plate pre-plated with ARPE-19 cells was discarded, and the serum mixtures were added, respectively, and cultured at 37° C. for 1 h, and then the supernatant was discarded. DMEM/F12 medium was added to continue culture, and the cell pathological changes were observed after 48 hours. When obvious lesion cells could be observed, the cells in the 24-well plate were fixed and permeabilized in a conventional manner, and incubated with gE-specific enzyme-labeled antibody (1B11-HPR, 1/2000 dilution; prepared by our laboratory) for immunoadsorption. After rinsing with PBST three times, ELISPOT color development was performed, and the cell plate was photographed using a fluorescent spot analyzer. The lesion spots in the photos were counted and the neutralization titer of the serum was calculated. In the experiment, the control well was not added with the serum samples to be tested, and the number of lesion spots in the well was the number of unneutralized viruses; the neutralization titer of serum antibody was defined as the maximum dilution at which the serum could neutralize 50% of the virus. The neutralization titer results were shown in FIG. 7. The results showed that mice immunized with gE (31-358) protein combined with risedronate adjuvant or AS01_{B} adjuvant could induce the production of neutralizing antibodies in the serum; in which, the titer of neutralizing antibodies could reach a high level of 103 after the booster immunization.

### Investigation of Half Effective Dose (ED₅₀) of gE (31-358)

In this experiment, the immunogenicity of gE (31-358) protein was investigated by determining the half effective dose (ED₅₀). The experimental animals were 3-4-week-old female BALB/c mice. The gE (31-358) protein prepared in Example 2 was adsorbed on aluminum adjuvant, and the protein concentrations were 1.00 μg/mL, 0.50 μg/mL, 0.25 μg/mL, 0.125 μg/mL, 0.0625 μg/mL, and 0.03125 μg/mL, respectively, that was, there were 6 dose groups in total. In each group, 6 BALB/c mice were intraperitoneally injected with 1 mL of the above concentration once. In addition, a blank group was set up, containing 6 BALB/c mice. Serum was collected in the fourth week after the injection, and varicella-zoster virus IgG detection kit (enzyme-linked immunosorbent assay, EIA) (National Medical Device Registration No.: 20173403325) was used to detect varicella-zoster virus IgG according to the instructions.

For the EIA detection results, the mean of the negative control well A value (NC) (if there was 1 well of negative control having A value of less than 0.80, it should be discarded; and if there were 2 wells of negative control both having A value of less than 0.80, the experiment should be repeated)×50% was calculated and used as CUTOFF value. All BALB/c mice were negative for VZV antibody before the injection. The detection results were shown in Table 2.

TABLE 2
ED₅₀ results of EIA detection of gE(31-358) protein in BALB/c mice

| Concentration, | Total number | Number of positives | VZV antibody |
|---|---|---|---|
| μg/mL | or mice | in 4 weeks | positive rate (%) |
| 1.00 | 6 | 5 | 96.43% |
| 0.50 | 6 | 6 | 95.65% |
| 0.25 | 6 | 6 | 94.12% |
| 0.125 | 6 | 6 | 90.91% |
| 0.0625 | 6 | 3 | 50.00% |
| 0.03125 | 6 | 1 | 10.00% |

ED₅₀ was calculated according to the Reed-Muench method. After 4 weeks of observation after the vaccine injection, blood was collected to detect ED₅₀. The results showed that the ED₅₀ of gE (31-358) protein was 0.063 μg, which indicated that a high level of immune antibodies could be produced at this dose.

Evaluation of gE-Specific Cellular Immune Response (Flow Cytometry)

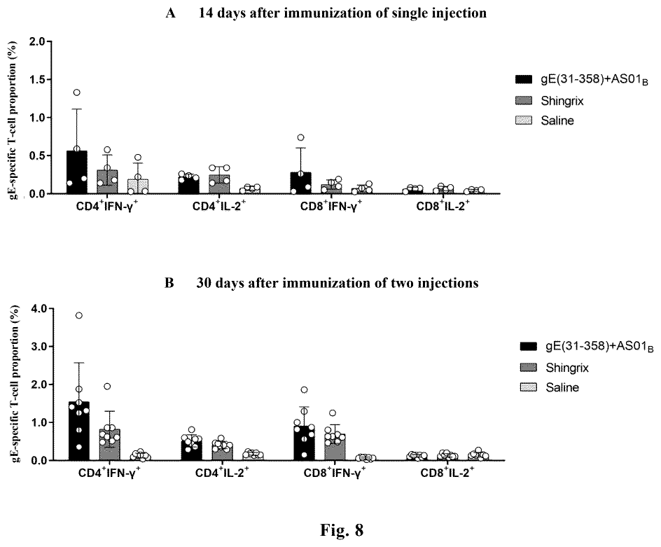

The mice used in this experiment were female, 6-week-old C57 mice. The gE (31-358) protein prepared by the method of Example 2 was injected into the tibialis muscle of the mice for immunization using AS01_{B} adjuvant, with an injection volume of 0.05 mL and a dose of 5 ug. The control group was immunized with the same dose of Shingrix vaccine (GSK), and the blank group was immunized with saline. The primary immunization was performed at week 0, and the booster immunization was performed at week 4. Four and eight mice were killed at week 2 (14 days after single injection) and week 8 (30 days after two injections), respectively. The spleen was taken out under sterile conditions, and a spleen cell suspension was prepared after grinding, filtering, and lysing red blood cells, and plated into a 96-well U-bottom plate at a density of 2×10⁶ cells/well. The gE peptide mixture library (1.25 μg/mL; which was an overlapping peptide library with a length of 15 aa, overlapping 11 aa, and covering aa 22-537 of gE protein and synthesized by Sangon Biotech (Shanghai) Co., Ltd.) was added to the culture medium as a stimulator. After 18 hours of culture, a Golgi inhibitor was added and cultured for another 6 hours. After the stimulated cells were washed, fixed, permeabilized, and incubated with a specific antibody labeled with fluorescent dye, flow cytometry was performed using a BD LSRFortessa™ X-20 cell analyzer to analyze the expression levels of cytokines such as IFN-γ and IL-2 in CD4⁺ and CD8⁺ cell subsets. The results were shown in FIG. 8. It could be seen that the combination of gE (31-358) protein and AS01_{B} adjuvant could stimulate the mice to produce a specific cellular immune response that was equivalent to that of Shingrix vaccine.

The results of this example showed that the gE (31-358) protein obtained in Example 2 can be mixed with an adjuvant to formulate into a vaccine with good immunogenicity, could induce high titer binding antibodies, neutralizing antibodies and specific cellular immune responses in animals, and could be used as a vaccine to prevent VZV primary infection and recurrent infection.

Although the specific embodiments of the present application have been described in detail, those skilled in the art will understand that various modifications and changes can be made to the details based on all the teachings that have been disclosed, and these changes are within the scope of protection of the present application. All of the present application is given by the appended claims and any equivalents thereof.

What is claimed is:

1. A truncated varicella-zoster virus (VZV) gE protein or variant thereof, wherein the truncated VZV gE protein has a truncation at the C-terminal of 75-445 amino acids as compared to the wild-type VZV gE protein; the variant has an amino acid sequence identity of at least 90%, such as at least 95%, at least 96%, at least 97%, at least 98%, at least 99%, or has a substitution (preferably, conservative substitution), addition or deletion of one or several (e.g., 1, 2, 3, 4, 5, 6, 7, 8 or 9) amino acids, as compared to the truncated VZV gE protein, and retains the biological function of the truncated VZV gE protein (e.g., the ability to induce a neutralizing antibody against VZV, and/or soluble expression in Escherichia coli);
preferably, as compared to the wild-type VZV gE protein, the truncated VZV gE protein has a truncation at the C-terminal of at most 445 amino acids, such as at most 442, at most 440, at most 430, at most 420, at most 410, at most 400, at most 390, at most 380, at most 370, at most 360, at most 350, at most 340, at most 330, at most 325 or at most 320 amino acids, for example, at most 330 amino acids; and/or, has a truncation at the C-terminal of at least 75 amino acids, such as at least 77, at least 80, at least 85, at least 90, at least 100, at least 110, at least 120, at least 130, at least 140, at least 150, at least 160, at least 170, at least 180, at least 190, at least 193, at least 200, at least 210, at least 220, at least 222, at least 230, at least 240, at least 248, at least 250, at least 260 or at least 265 amino acids, for example at least 260 amino acids;
preferably, as compared to the wild-type VZV gE protein, the truncated VZV gE protein has a truncation at the C-terminal of 80-445, 190-445, 220-445, 245-445, 260-445, 75-330, 80-330, 190-330, 220-330, 245-330, 260-330, 75-310, 80-310, 190-310, 220-310, 245-310, 260-310, 75-300, 80-300, 190-300, 220-300, 245-300, 260-300, 75-270, 80-270, 190-270, 220-270, 245-270, 77-442, 85-442, 193-442, 222-442, 248-442, 265-442, 77-320, 85-320, 193-320, 222-320, 248-320, 265-320, 77-303, 85-303, 193-303, 222-303, 248-303, 265-303, 77-293, 85-293, 193-293, 222-293, 248-293, 265-293, 77-265, 85-265, 193-265, 222-265 or 248-265 amino acids.

2. The truncated VZV gE protein or variant thereof according to claim 1, wherein, compared to the wild-type VZV gE protein, the truncated VZV gE protein has no truncation at the N-terminal or has a truncation at the N-terminal of 1-170 amino acids;
preferably, compared to the wild-type VZV gE protein, the truncated VZV gE protein has a truncation at the N-terminal of at most 170 amino acids, such as at most 165, at most 160, at most 155, at most 150, at most 145, at most 140, at most 139, at most 135, at most 130 or at most 127 amino acids, for example at most 130 amino acids; and/or, has a truncation at the N-terminal of at least 1 amino acid, such as at least 5, at least 10, at least 15, at least 20, at least 25 or at least 30 amino acids, such as at least 30 amino acids;
preferably, compared to the wild-type VZV gE protein, the truncated VZV gE protein has a truncation at the N-terminal of 20-170, 1-155, 20-155, 1-140, 20-140, 1-130, 20-130, 1-85, 20-85, 1-75, 20-75, 1-30, 20-30, 30-165, 30-152, 30-139, 30-127, 30-80, 30-73, 30-75, 30-85, 30-130, 30-140, 30-155 or 30-170 amino acids.

3. The truncated VZV gE protein or variant thereof according to claim 1 or 2, wherein, compared to the wild-type VZV gE protein, the truncated VZV gE protein has a truncation at the C-terminal of 260-330 amino acids, for example, 260-310, 260-300, 265-320, 265-303, 265-293 amino acids;
preferably, compared to the wild-type VZV gE protein, the truncated VZV gE protein has no truncation at the N-terminal or has a truncation at the N-terminal of 1-130 amino acids, for example, 20-130, 20-85, 20-75, 20-30, 30-127, 30-80, 30-73, 30-75, 30-85, 30-130 amino acids;
preferably, compared to the wild-type VZV gE protein, the truncated VZV gE protein has a truncation at the C-terminal of 260-330 amino acids, for example, 260-310, 260-300, 265-320, 265-303, 265-293 amino acids; has no truncation at the N-terminal or has a truncation at the N-terminal of 20-130 amino acids, for example, 20-85, 20-75, 20-30, 30-127, 30-80, 30-73, 30-75, 30-85, 30-130 amino acids;
preferably, compared to the wild-type VZV gE protein, the truncated VZV gE protein has a truncation at the C-terminal of 77, 85, 193, 222, 248, 265, 293, 303 or 320 amino acids; has no truncation at the N-terminal or a truncation at the N-terminal of 30, 73, 80, 127 or 139 amino acids;
preferably, compared to the wild-type VZV gE protein, the truncated VZV gE protein has a truncation at the C-terminal of 265, 293, 303 or 320 amino acids; has a truncation at the N-terminal of 30, 73, 80 or 127 amino acids;
preferably, compared to the wild-type VZV gE protein, the truncated VZV gE protein has a truncation at the C-terminal of 265 amino acids; has a truncation at the N-terminal of 30 amino acids.

4. The truncated VZV gE protein or variant thereof according to any one of claims 1 to 3, wherein the wild-type VZV gE protein has an amino acid sequence as shown in SEQ ID NO: 19.

5. The truncated VZV gE protein or variant thereof according to any one of claims 1 to 4, wherein the truncated VZV gE protein has an amino acid sequence as shown in any one of SEQ ID NOs: 1-6, 20-30.

6. An isolated nucleic acid molecule, which encodes the truncated VZV gE protein or variant thereof according to any one of claims 1 to 5.

7. A vector, which comprises the isolated nucleic acid molecule according to claim 6.

8. A host cell, which comprises the isolated nucleic acid molecule according to claim 6 or the vector according to claim 7;
preferably, the host cell is selected from the group consisting of prokaryotic cell (e.g., Escherichia coli cell), and eukaryotic cell (e.g., yeast cell, insect cell, plant cell, mammalian cell);
preferably, the host cell is an Escherichia coli cell.

9. A method for preparing the truncated VZV gE protein or variant thereof according to any one of claims 1 to 5, comprising culturing the host cell according to claim 8 under a condition that allows protein expression, and recovering the truncated VZV gE protein or variant thereof from a culture of the cultured host cell;
preferably, the method comprises recovering the truncated VZV gE protein or variant thereof from the culture supernatant or lysis supernatant of the cultured host cell;
preferably, the host cell is Escherichia coli.

10. The method according to claim 9, wherein the recovery step comprises protein purification;
preferably, the protein purification comprises performing an ion exchange chromatography, hydroxyapatite chromatography, and/or hydrophobic interaction chromatography;
preferably, the ion exchange chromatography comprises:
a) allowing the truncated VZV gE protein or variant thereof to bind to an anion exchange chromatography medium (e.g., Q Sepharose 4 Fast Flow) in a solution with a pH of 7.5 to 8.5 (e.g., pH 8.0) and a salt concentration of 0 mM to 200 mM (e.g., 0 mM to 50 mM);
b) performing a gradient elution by gradually increasing the salt concentration of the solution; and
c) collecting an elution fraction containing the truncated VZV gE protein or variant thereof when the solution salt concentration is within the range of 350 mM to 450 mM (e.g., at 400 mM).

11. An immunogenic composition, which comprises the truncated VZV gE protein or variant thereof according to any one of claims 1 to 5, and optionally a pharmaceutically acceptable carrier and/or excipient (e.g., adjuvant);
preferably, the immunogenic composition comprises the truncated VZV gE protein or variant thereof according to any one of claims 1 to 5 and an adjuvant, wherein the adjuvant is selected from the group consisting of: risedronate adjuvant (e.g., zinc-aluminum hybrid adjuvant containing risedronate sodium), aluminum adjuvant (e.g., aluminum hydroxide adjuvant, aluminum phosphate adjuvant), oil emulsion adjuvant, cytokine, TLR agonist, CpG adjuvant, liposome, AS01_{B} adjuvant, zoledronate sodium, monophosphoryl lipid A (MPL), cholesterol-containing liposome and combination thereof; preferably, the adjuvant is selected from the group consisting of: risedronate adjuvant (e.g., zinc-aluminum hybrid adjuvant containing risedronate sodium), aluminum adjuvant (e.g., aluminum hydroxide adjuvant, aluminum phosphate adjuvant), AS01_{B} adjuvant and combination thereof;
preferably, the immunogenic composition comprises the truncated VZV gE protein or variant thereof according to claim 1 or 2 and AS01_{B} adjuvant;
preferably, the immunogenic composition is a vaccine.

12. Use of the truncated VZV gE protein or variant thereof according to any one of claims 1 to 5, or the isolated nucleic acid molecule according to claim 6, or the vector according to claim 7, or the host cell according to claim 8 in the manufacture of an immunogenic composition, wherein the immunogenic composition is used for inducing an immune response against VZV in a subject and/or for preventing and/or treating a VZV infection or a disease associated with VZV infection in a subject;
preferably, the immunogenic composition is a vaccine;
preferably, the VZV infection is a primary infection or a recurrent infection of VZV;
preferably, the disease associated with VZV infection is selected from the group consisting of: herpes zoster, varicella, and postherpetic neuralgia;
preferably, the subject is a mammal, such as a human.

13. A method for inducing an immune response against VZV in a subject and/or for preventing and/or treating a VZV infection or a disease associated with VZV infection in a subject, comprising: administering an effective amount of the truncated VZV gE protein or variant thereof according to any one of claims 1 to 5, the isolated nucleic acid molecule according to claim 6, the vector according to claim 7, the host cell according to claim 8, or the immunogenic composition according to claim 11 to the subject in need thereof;
preferably, the VZV infection is a primary infection or a recurrent infection of VZV;
preferably, the disease associated with VZV infection is selected from the group consisting of: herpes zoster, varicella, and postherpetic neuralgia;
preferably, the subject is a mammal, such as a human.

14. A method for detecting the presence of VZV gE protein-specific antibody in a sample, comprising using the truncated VZV gE protein or variant thereof according to any one of claims 1 to 5;
preferably, the method is an immunological detection, such as immunoblotting, enzyme immunoassay (e.g., ELISA), chemiluminescent immunoassay, fluorescent immunoassay or radioimmunoassay;
preferably, the method comprises: (1) contacting the sample with the truncated VZV gE protein or variant thereof according to any one of claims 1 to 5; (2) detecting the formation of a protein-antibody immune complex or detecting an amount of the immune complex; the formation of the immune complex indicates the presence of VZV gE protein-specific antibody in the sample;
preferably, the method further comprises using a second antibody with a detectable label (e.g., an enzyme (e.g., horseradish peroxidase or alkaline phosphatase), a chemiluminescent agent (e.g., acridinium ester compound, luminol and derivative thereof, or ruthenium derivative), a fluorescent dye (e.g., fluorescein or fluorescent protein), a radionuclide or a biotin) to detect the presence of VZV gE protein-specific antibody in the sample;
preferably, the second antibody is specific for a constant region contained in an antibody of the species (e.g., human) from which the sample to be tested comes;
preferably, the second antibody is an anti-immunoglobulin (e.g., human immunoglobulin) antibody, such as an anti-IgG antibody;
preferably, the sample is a body fluid sample (e.g., whole blood, plasma, serum, salivary excretion or urine) from a subject (e.g., a mammal, preferably a human).

15. Use of the truncated VZV gE protein or variant thereof according to any one of claims 1 to 5, or the isolated nucleic acid molecule according to claim 6, or the vector according to claim 7, or the host cell according to claim 8 in the manufacture of a detection reagent, wherein the detection reagent is used to detect the presence of VZV gE protein-specific antibody in a sample;
preferably, the detection reagent detects the presence of VZV gE protein-specific antibody in the sample by the method according to claim 14;
preferably, the sample is a body fluid sample (e.g., whole blood, plasma, serum, salivary excretion or urine) from a subject (e.g., a mammal, preferably a human).

16. A kit, which comprises the truncated VZV gE protein or variant thereof according to any one of claims 1 to 5, or the isolated nucleic acid molecule according to claim 6, or the vector according to claim 7, or the host cell according to claim 8;
preferably, the kit comprises the truncated VZV gE protein or variant thereof according to any one of claims 1 to 5, and a second antibody, wherein the second antibody is as defined in claim 14.


SEQUENCE LISTING (31 sequences)

SEQ ID NO: 1 - protein, 273 aa, synthetic construct
  source 1..273: note = gE(31-303) amino acid sequence
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNM

SEQ ID NO: 2 - protein, 290 aa, synthetic construct
  source 1..290: note = gE(31-320) amino acid sequence
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWK

SEQ ID NO: 3 - protein, 328 aa, synthetic construct
  source 1..328: note = gE(31-358) amino acid sequence
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFS

SEQ ID NO: 4 - protein, 278 aa, synthetic construct
  source 1..278: note = gE(81-358) amino acid sequence
YIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFS

SEQ ID NO: 5 - protein, 231 aa, synthetic construct
  source 1..231: note = gE(128-358) amino acid sequence
DDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFS

SEQ ID NO: 6 - protein, 191 aa, synthetic construct
  source 1..191: note = gE(140-330) amino acid sequence
GDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTP

SEQ ID NO: 7 - DNA, 819 bp, synthetic construct
  source 1..819: mol_type = other DNA; note = gE(31-303) encoding nucleotide sequence
tccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatg

SEQ ID NO: 8 - DNA, 870 bp, synthetic construct
  source 1..870: mol_type = other DNA; note = gE(31-320) encoding nucleotide sequence
tccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaa

SEQ ID NO: 9 - DNA, 984 bp, synthetic construct
  source 1..984: mol_type = other DNA; note = gE(31-358) encoding nucleotide sequence
tccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagc

SEQ ID NO: 10 - DNA, 834 bp, synthetic construct
  source 1..834: mol_type = other DNA; note = gE(81-358) encoding nucleotide sequence
tatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagc

SEQ ID NO: 11 - DNA, 693 bp, synthetic construct
  source 1..693: mol_type = other DNA; note = gE(128-358) encoding nucleotide sequence
gacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagc

SEQ ID NO: 12 - DNA, 573 bp, synthetic construct
  source 1..573: mol_type = other DNA; note = gE(140-330) encoding nucleotide sequence
ggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgccc

SEQ ID NO: 13 - DNA, 20 bp, synthetic construct
  source 1..20: mol_type = other DNA; note = Primer gE-1-F nucleotide sequence
gggacagttaataaacctgt

SEQ ID NO: 14 - DNA, 20 bp, synthetic construct
  source 1..20: mol_type = other DNA; note = Primer gE-546-R nucleotide sequence
tgcaagccctccggtccatg

SEQ ID NO: 15 - DNA, 40 bp, synthetic construct
  source 1..40: mol_type = other DNA; note = Primer B11-gE-31-F nucleotide sequence
aagaaggagatatacatatgtccgtcttgcgatacgatga

SEQ ID NO: 16 - DNA, 40 bp, synthetic construct
  source 1..40: mol_type = other DNA; note = Primer B11-gE-358-R nucleotide sequence
ttgttagcagccggatctcagctaaacgtatcaccaactg

SEQ ID NO: 17 - DNA, 20 bp, synthetic construct
  source 1..20: mol_type = other DNA; note = Primer B11-NdeI-VF nucleotide sequence
catatgtatatctccttctt

SEQ ID NO: 18 - DNA, 20 bp, synthetic construct
  source 1..20: mol_type = other DNA; note = Primer B11- TGA-VR nucleotide sequence
tgagatccggctgctaacaa

SEQ ID NO: 19 - protein, 623 aa, synthetic construct
  source 1..623: note = amino acid sequence of gE in full-length
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 20 - protein, 300 aa, synthetic construct
  source 1..300: note = gE(31-330) amino acid sequence
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTP

SEQ ID NO: 21 - protein, 230 aa, synthetic construct
  source 1..230: note = gE(74-303) amino acid sequence
AYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNM

SEQ ID NO: 22 - protein, 247 aa, synthetic construct
  source 1..247: note = gE(74-320) amino acid sequence
AYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWK

SEQ ID NO: 23 - protein, 257 aa, synthetic construct
  source 1..257: note = gE(74-330) amino acid sequence
AYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTP

SEQ ID NO: 24 - protein, 285 aa, synthetic construct
  source 1..285: note = gE(74-358) amino acid sequence
AYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFS

SEQ ID NO: 25 - protein, 223 aa, synthetic construct
  source 1..223: note = gE(81-303) amino acid sequence
YIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNM

SEQ ID NO: 26 - protein, 240 aa, synthetic construct
  source 1..240: note = gE(81-320) amino acid sequence
YIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWK

SEQ ID NO: 27 - protein, 250 aa, synthetic construct
  source 1..250: note = gE(81-330) amino acid sequence
YIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTP

SEQ ID NO: 28 - protein, 176 aa, synthetic construct
  source 1..176: note = gE(128-303) amino acid sequence
DDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNM

SEQ ID NO: 29 - protein, 193 aa, synthetic construct
  source 1..193: note = gE(128-320) amino acid sequence
DDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWK

SEQ ID NO: 30 - protein, 203 aa, synthetic construct
  source 1..203: note = gE(128-330) amino acid sequence
DDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTP

SEQ ID NO: 31 - protein, 515 aa, synthetic construct
  source 1..515: note = gE(Bac) amino acid sequence
RITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLR